(19) United States
(12) Patent Application Publication
(10) Pub. No.: US 2007/0276030 A1
(43) Pub. Date: Nov. 29, 2007

(54) Pyranobenzothiophene derivatives to treat infection with hepatitis c virus

(75) Inventor: Ariamala Gopalsamy, 1106 DEVON COURT MAHWAK (US)

Correspondence Address: FITZPATRICK CELLA (WYETH), 30 ROCKEFELLER PLAZA, NEW YORK, NY, 10112-3800, US

(73) Assignees: WYETH, MADISON NEW JERSEY, NJ (US); VIROPHARMA INCORPORATED, EXTON, PENNSYLVANIA, PA (US)

(21) Appl. No.: 10/567,649

(22) PCT Filed: Aug. 13, 2004
(86) PCT No.: PCT/US04/26504

§ 371 Date: Oct. 4, 2006

Related U.S. Application Data
(60) Provisional application No. 60/494,789, filed on Aug. 14, 2003.

(51) Int. Cl. A61K 31/381; C07D 495/02

(52) U.S. Cl. 514/443; 549/43

(57) ABSTRACT

The invention is directed to a compound of the formula: 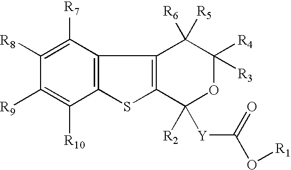
wherein substitutions fat R¹, R², R³-R¹², and Y are set forth in the specification; pharmaceutical compositions comprising said compound, methods of treating or preventing a Hepatitis C viral infection in a mammal comprising contacting the mammal with an effective amount of said compound or pharmaceutical compositions including said compound and methods of inhibiting replication of a Hepatitis C virus comprising contacting the HCV virus with an effective amount of said compound or pharmaceutical compositions including said compound.

### BACKGROUND OF THE INVENTION

Hepatitis C is a common viral infection that can lead to chronic Hepatitis, cirrhosis, liver failure, and hepatocellular carcinoma. Infection with the Hepatitis C virus (HCV) leads to chronic Hepatitis in at least 85% of cases, is the leading reason for liver transplantation, and is responsible for at least 10,000 deaths annually in the United States (Hepatology, 1997, 26 (Suppl. 1), 2S-10S).

The Hepatitis C virus is a member of the Flaviviridae family, and the genome of HCV is a single-stranded linear RNA of positive sense (Id. At 11S-14S). HCV displays extensive genetic heterogeneity, at least 6 genotypes and more than 50 subtypes have been identified.

There is no effective vaccine to prevent HCV infection. The only therapy currently available is treatment with interferon-α (INF-α) or combination therapy of INF-α with the nucleoside analog ribavirin (Antiviral Chemistry and Chemotherapy, 1997, 8, 281-301). However, only about 40% of treated patients develop a sustained response, so there is a need for more effective anti-HCV therapeutic agents.

The HCV genome contains a number of non-structural proteins: NS2, NS3, NS4A, NS4B, NS5A, and NS5B (J. General Virology, 2000, 81, 1631-1648). NS5B is an RNA-dependent RNA polymerase which is essential for viral replication, and therefore, the inhibition of NS5B is a suitable target for the development of therapeutic agents.

In U.S. Pat. No. 4,021,451 (May 3, 1977) a process applicable to the preparation of a wide variety of novel heterocycles having a newly formed pyran ring is disclosed including [p-(3,4-dihydro-1-methyl-1H-[1]benzothieno[2,3-c]pyran-1-yl)phenyl]-acetic acid, but the compound lacks any biological activity such as antiinflammatory or antibacterial or antifungal activity. A publication in Scientia Pharmaceutica (67, 97-102, 1999) discloses the preparation of 1-alkyl-3, 4-dihydro-benzo[b]thienopyran acids. But these compounds with no aromatic substituents were found to have no antiphlogistic effect.

### BRIEF SUMMARY OF THE INVENTION

This invention relates to pyranobenzothiophene derivatives, pharmaceutical compositions comprising pyranobenzothiophene derivatives, processes for their preparation, and to their use in the treatment of Hepatitis C viral infection.

In accordance with this invention there is provided a group of compounds represented by formula (I): 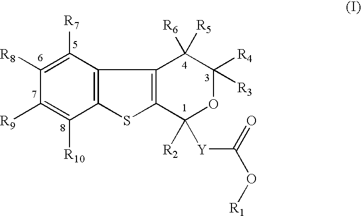
wherein:

R₁ is H, a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, an alkynyl of 2 to 7 carbon atoms, or an arylalkyl or an alkylaryl of 7 to 12 carbon atoms;

R₂ is H, a straight chain alkyl of 1 to 12 carbon atoms, a branched alkyl of 3 to 12 carbon atoms, a cycloalkyl of 3 to 12 carbon atoms, an alkenyl of 2 to 7 carbon atoms, an alkynyl of 2 to 7 carbon atoms, an alkoxyalkyl or alkoxycarbonyl of 2 to 12 carbon atoms, an arylalkyl or alkylaryl of 7 to 12 carbon atoms, a cyanoalkyl of 1 to 8 carbon atoms, an alkylthioalkyl of 2 to 16 carbon atoms, a cycloalkyl-alkyl of 4 to 24 carbon atoms, a substituted or unsubstituted aryl, or a heteroaryl;

R₃-R₆ are independently H, a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, a substituted or unsubstituted aryl, furanylmethyl, arylalkyl or alkylaryl of 7 to 12 carbon atoms, alkynyl of 2 to 7 carbon atoms, or R₅ and R₆ together with the ring carbon atom to which they are attached form a carbonyl group;

R₇-R₁₀ are independently H, a straight chain alkyl of 1 to 8 carbon atoms, a branched alkyl of 3 to 12 carbons atoms, a cycloalkyl of 3 to 12 carbon atoms, an alkenyl of 2 to 7 carbon atoms, a substituted or unsubstituted aryl, a substituted or unsubstituted heteroaryl, furanylmethyl, arylalkyl or alkylaryl of 7 to 12 carbon atoms, alkynyl of 2 to 7 carbon atoms, phenylalkynyl, alkoxy of 1 to 8 carbon atoms, arylalkoxy of 7 to 12 carbon atoms, alkylthio of 1 to 8 carbon atoms, trifluoromethoxy, trifluoroethoxy, trifluoromethylthio, trifluoroethylthio, acyl of 1 to 7 carbon atoms, COOH, COO-alkyl, CONR₁₁R₁₂, F, Cl, Br, I, CN, CF₃, NO₂, alkylsulfinyl of 1 to 8 carbon atoms, alkylsulfonyl of 1 to 6 carbon atoms, pyrrolidinyl, or thiazolidinyl;

R₁₁-R₁₂ are independently H, straight chain alkyl of 1 to 8 carbon atoms, branched alkyl of 3 to 12 carbon atoms, cycloalkyl of 3 to 12 carbon atoms, a substituted or unsubstituted aryl or heteroaryl;

Y is (CH₂)ₙ and n is an integer from 0 to 3, aryl or heteroaryl, cycloalkyl or heterocycloalkyl or R₂ and Y together with the ring carbon atom to which they are attached form a substituted or unsubstituted spirocyclic cycloalkyl ring of 3 to 8 carbon atoms; or a crystalline form or a pharmaceutically acceptable salt thereof.

For purposes of this invention the term “alkyl” includes either straight or branched alkyl moieties. The length of a straight alkyl moiety can be from 1 to 12 carbon atoms, but is preferably 1 to 8 carbon atoms. Branched alkyl moieties can contain 3 to 12 carbon atoms. These alkyl moieties may be unsubstituted or substituted. The term “alkenyl” refers to a substituted or unsubstituted radical aliphatic hydrocarbon containing one double bond and includes alkenyl moieties of both straight, preferably of 2 to 7 carbon atoms and branched, preferably of 3 to 7 carbon atoms. Such alkenyl moieties may exist in the E or Z configurations; the compounds of this invention include both configurations. The term “alkynyl” includes substituted and unsubstituted alkynyl moieties of both straight chain containing 2 to 7 carbon atoms and branched containing 4 to 7 carbon atoms having at least one triple bond. The term “cycloalkyl” refers to substituted or unsubstituted alicyclic hydrocarbon groups having 3 to 12 carbon atoms and includes but is not limited to: cyclopropyl, cyclobutyl, cyclopentyl, cyclohexyl, cycloheptyl, norbornyl, or adamantyl. For purposes of this invention the term “aryl” is defined as an aromatic hydrocarbon moiety and may be substituted or unsubstituted. An aryl may be selected from but not limited to, the group consisting of: phenyl, α-naphthyl, β-naphthyl, biphenyl, anthryl, tetrahydronaphthyl, phenanthryl, fluorenyl, indanyl, biphenylenyl, acenaphthenyl, acenaphthylenyl, or phenanthrenyl groups. In one embodiment the substituted aryl, heteroaryl, cycloalkyl, heterocycloalkyl, spirocyclic cycloalkyl, or spirocyclic heterocycloalkyl may be optionally mono-, di-, tri- or tetra-substituted with substituents selected from, but not limited to, the group consisting of alkyl, acyl, alkoxycarbonyl, alkoxy, alkoxyalkyl, alkoxyalkoxy, cyano, halogen, hydroxy, nitro, trifluoromethyl, trifluoromethoxy, trifluoropropyl, amino, alkylamino, dialkylamino, dialkylaminoalkyl, hydroxyalkyl, alkylthio, —SO₃H, —SO₂NH₂, —SO₂NHalkyl, —SO₂N(alkyl)₂, —CO₂H, CO₂NH₂, CO₂NHalkyl, and —CO₂N(alkyl)₂. Preferred substituents for aryl, heteroaryl, cycloalkyl, heterocycloalkyl, spirocyclic cycloalkyl, and spirocyclic heterocycloalkyl include but are not limited to: alkyl, halogen, amino, alkylamino, dialkylamino, trifluoromethyl trifluoromethoxy, arylalkyl, and alkylaryl.

For purposes of this invention the term “heteroaryl” is defined as an aromatic heterocyclic ring system (monocyclic or bicyclic) and may be substituted or unsubstituted where the heteroaryl moieties are five or six membered rings containing 1 to 4 heteroatoms selected from the group consisting of S, N, and O, and include but are not limited to: (1) furan, thiophene, indole, azaindole, oxazole, thiazole, isoxazole, isothiazole, imidazole, N-methylimidazole, pyridine, pyrimidine, pyrazine, pyrrole, N-methylpyrrole, pyrazole, N-methylpyrazole, 1,3,4-oxadiazole, 1,2,4-triazole, 1-methyl-1,2,4-triazole, 1H-tetrazole, 1-methyltetrazole, benzoxazole, benzothiazole, benzofuran, benzisoxazole, benzimidazole, N-methylbenzimidazole, azabenzimidazole, indazole, quinazoline, quinoline, pyrrolidinyl; (2) a bicyclic aromatic heterocycle where a phenyl, pyridine, pyrimidine or pyridizine ring is: (i) fused to a 6-membered aromatic (unsaturated) heterocyclic ring having one nitrogen atom; (ii) fused to a 5 or 6-membered aromatic (unsaturated) heterocyclic ring having two nitrogen atoms; (iii) fused to a 5-membered aromatic (unsaturated) heterocyclic ring having one nitrogen atom together with either one oxygen or one sulfur atom; or (iv) fused to a 5-membered aromatic (unsaturated) heterocyclic ring having one heteroatom selected from O, N or S.

For purposes of this invention the term “heterocycloalkyl” refers to a substituted or unsubstituted alicyclic ring system (moncyclic or bicyclic) wherein the heterocycloalkyl moieties are 3 to 12 membered rings containing 1 to 6 heteroatoms selected from the group consisting of S, N, and O.

For the purposes of this invention the term “spirocyclic heterocycloalky” refers to a substituted or unsubstituted spirocyclic heterocyclic ring system wherein the spirocyclic heterocycloalkyl moieties are 3 to 12 membered rings containing 1 to 6 heteroatoms selected from the group consisting of S, N, and O.

For the purposes of this invention the term “alkoxy” is defined as C₁-C₁₂alkyl-O—; the term “aryloxy” is defined as aryl-O—; the term “heteroaryloxy” is defined as heteroaryl-O—; the term “cycloalkyloxy” is defined as cycloalkyl-O—; the term “heterocycloalkyloxy” is defined as heterocycloalkyl-O—; wherein alkyl, aryl, heteroaryl, cycloalkyl, and heterocycloalkyl are as defined above.

For purposes of this invention the term “arylalkyl” is defined as aryl-C₁-C₆-alkyl, preferably the arylalkyl moiety is comprised of 7-12 carbon atoms. Arylalkyl moieties include benzyl, 1-phenylethyl, 2-phenylethyl, 3-phenylpropyl, 2-phenylpropyl and the like.

For purposes of this invention the term “alkylaryl” is defined as C₁-C₈-alkyl-aryl. Preferably the alkylaryl moiety is comprised of 7-12 carbon atoms.

For purposes of this invention the term “alkylthio” is defined as C₁-C₈-alkyl-S—.

For purposes of this invention “alkoxyalkyl,” “cycloalkyl-alkyl,” “alkylthioalkyl,” “cycloalkyloxy-alkyl,” “aryloxyalkyl,” “heteroaryloxyalkyl,” and “heterocycloalkyl-alkyl,” “heterocycloalkyloxy-alkyl denote an alkyl group as defined above that is further substituted with an alkoxy, cycloalkyl, alkylthio, cycloalkyloxy; aryloxy, heteroaryloxy, heterocycloalkyl, or heterocycloalkyloxy group as defined above.

For purposes of this invention “arylalkoxy,” “alkoxyalkoxy,” “alkylthioalkoxy,” and “heteroarylalkoxy” denote an alkoxy group as defined above that is further substituted with an aryl, alkoxy, alkylthio, or heteroaryl group as defined above.

For purposes of this invention “arylthio” and “heteroarylthio,” denote a thio group that is further substituted with an aryl or heteroaryl group as defined above.

For purposes of this invention “arylthioalkyl” and “heteroarylthioalkyl” denote an alkyl group as defined above that is further substituted with an arylthio or heteroarylthio group as defined above.

For purposes of this invention the term “arylalkylthio” is defined as aryl-C₁-C₈-alkyl-S—; “heteroarylalkylthio” is defined as heteroaryl-C₁-C₈-alkyl-S—, where aryl and heteroaryl are as defined above.

For purposes of this invention “aryloxyalkylthio” is defined as aryloxy-C₁-C₈-alkyl-S; “heteroaryloxyalkylthio” is defined as heteroaryloxy-C₁-C₈-alkyl-S—; where aryloxy, heteroaryloxy, and alkyl are defined above.

For purposes of this invention “phenylalkynyl” is an alkynyl group further substituted with a phenyl group.

The term “cyanoalkyl” refers to an alkyl radical, as defined above, that is further substituted with a cyano group. The preferred embodiment is wherein the alkyl radical contains 1 to 8 carbon atoms.

In the most preferred embodiment of this invention a substituted methyl comprises a methyl substituent further substituted with for example a furanyl group. In another embodiment of this invention a furanyl substituent is further substituted with a methyl group.

In a preferred embodiment of this invention trifluoromethoxy is CF₃O—. In another embodiment of this invention trifluoromethylthio is CF₃S—.

In one embodiment of this invention trifluoroethoxy includes but is not limited to CF₃CH₂O—. In another embodiment of this invention trifluoroethylthio includes but is not limited to CF₃CH₂S—.

The terms “monoalkylamino” and “dialkylamino” refer to moieties with one or two alkyl groups wherein the alkyl chain is 1 to 8 carbons and the groups may be the same or different. The terms monoalkylaminoalkyl and dialkylaminoalkyl refer to monoalkylamino and dialkylamino moieties with one or two alkyl groups (the same or different) bonded to the nitrogen atom which is attached to an alkyl group of 1 to 8 carbon atoms.

“Acyl” is a radical of the formula —(C═O)-alkyl or —(C═O)-perfluoroalkyl wherein the alkyl radical or perfluoroalkyl radical is 1 to 7 carbon atoms; preferred examples include but are not limited to, acetyl, propionyl, butyryl, trifluoroacetyl.

The term “carbonyl” or “oxo” refers to the radical —C(O)—.

For purposes of this invention the term “alkylsulfinyl” is defined as a R′SO— radical, where R′ is an alkyl radical of 1 to 8 carbon atoms. Alkylsulfonyl is a R′SO₂— radical, where R′ is an alkyl radical of 1 to 6 carbon atoms. Alkylsulfonamido, alkenylsulfonamido, alkynylsulfonamido are R′SO₂NH— radicals, where R′ is an alkyl radical of 1 to 8 carbon atoms, an alkenyl radical of 2 to 8 carbon atoms, or an alkynyl radical of 2 to 8 carbon atoms, respectively.

Saturated or partially saturated heteroaryl groups are defined in this invention as heterocyclic rings selected from but not limited to the moieties: azetidinyl, 1,4-dioxanyl, hexahydroazepinyl, piperazinyl, piperidinyl, pyrrolidinyl, morpholinyl, thiomorpholinyl, dihydrobenzimidazolyl, dihydrobenzofuranyl, dihydrobenzothienyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, dihydro-1,4-dioxanyl, tetrahydrofuranyl, tetrahydrothienyl, tetrahydroquinolinyl, and tetrahydroisoquinolinyl.

The term “substituent” is used herein to refer to an atom radical, a functional group radical or a moiety radical that replaces a hydrogen radical on a molecule. Unless expressly stated otherwise, it should assumed that any of the substituents may be optionally substituted with one or more groups selected from: alkyl, halogen, nitro, amino, hydroxyl, cyano, alkylamino, dialkylamino, alkoxy, haloalkoxy, alkylthio, mercapto, haloalkylthio, aryl, aryloxy, arylthio, heteroaryl, heteroaryloxy, heteroarylthio or acyl.

For the purposes of this invention the term “substituted” refers to where a hydrogen radical on a molecule has been replaced by another atom radical, a functional group radical or a moiety radical; these radicals being generally referred to as “substituents.”

For purposes of this invention, the term “BB7” denotes an RNA-dependent RNA polymerase hepatitis C virus protein sequence which is derived from HCV replicon. A discussion of BB7 and related technology can be found in Blight, K et al. (2000) Science 290:1972-1974. BB7 can be licensed from Apath, LLC (893 North Warson Road, Saint Louis Mo. 63141, USA). BB7 is also referred to as Con1 HCV sequence and discussions of Con1 can be found in the following references: Lohmann, V. et al. (1999) Science 285:110-113; Pietschmann, T. et al. (2001) J. Virol. 73:1252-1264; Lohmann, V. et al. (2001) J. Virol. 75:1437-1449.

The compounds of this invention may contain an asymmetric carbon atom and some of the compounds of this invention may contain one or more asymmetric centers and may thus give rise to stereoisomers, such as enantiomers and diastereomers. The stereoisomers of the instant invention are named according to the Cahn-Ingold-Prelog System. While shown without respect to stereochemistry in Formula (I), the present invention includes all the individual possible stereoisomers; as well as the racemic mixtures and other mixtures of R and S stereoisomers (scalemic mixtures which are mixtures of unequal amounts of enantiomers) and pharmaceutically acceptable salts thereof. It should be noted that stereoisomers of the invention having the same relative configuration at a chiral center may nevertheless have different R and S designations depending on the substitution at the indicated chiral center.

For compounds of this invention containing two chiral centers, four possible stereoisomers are possible; these four stereoisomers are classified as two racemic pairs of diastereomers. These compounds of the invention may be present as racemic diastereomers which would be designated following the convention described in the 1997 Chemical Abstracts Index Guide, Appendix IV (Columbus, Ohio) whereas the first cited chiral atom is designated R* and the next cited chiral atom is designated R* if it possesses the same chirality as the first cited stereocenter or S* if it possesses opposite chirality to the first cited stereocenter. Alternatively, these compounds of the invention may be present as non-racemic mixtures of two diastereomers owing to the existence of a predefined stereocenter. In these instances, the predefined stereocenter is assigned based on the Cahn-Ingold-Prelog System and the undefined stereocenter is designated R* to denote a mixture of both R and S stereoisomers at this center. Compounds of this invention which possess two chiral centers but which are present as single stereoisomers are described using the Cahn-Ingold-Prelog System.

Based on the chiral center at the C₁ carbon position in formula (I), a preferred embodiment of the instant invention is the compound of formula (Ia) shown below: 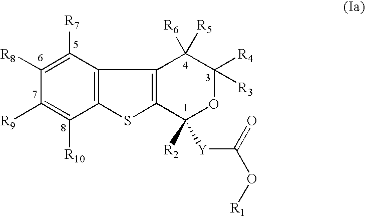
The configuration at C₁ in Formula (Ia) for purposes of this invention is also referred to as “Isomer A”, and the opposite configuration at C₁ is herein defined as “Isomer B” and has the formula (1b) shown below:

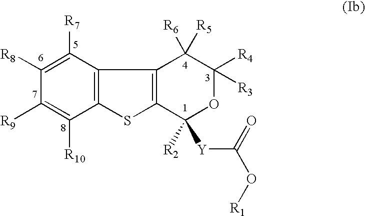

In one embodiment of this invention the compound of the invention is comprised of a ratio of Isomer A to Isomer B of greater than 1:1. In the most preferred embodiment the compound is comprised of 100% Isomer A. In further embodiments the compound is comprised of a ratio of Isomer A to Isomer B of at least about 9:1. In another embodiment the compound is comprised of a ratio of Isomer A to Isomer B of at least about 8:1. Additionally the compound is comprised of a ratio of Isomer A to Isomer B of at least about 7:1. The isomers described above may be pharmaceutically acceptable salts thereof.

The compounds of the current invention may be alkene diastereomers. The alkene diastereomers can be designated using the (E)-(Z) system. One skilled in the art will be familiar with this system of nomenclature. Where alkene compounds are disclosed without stereospecifity it is intended that both of the diastereomers are encompassed.

Pharmaceutically acceptable salts of the compounds of formula (I) having acidic moieties at R₃, R₄, R₅, R₆, R₇, R₈, R₉, or R₁₀ may be formed from organic and inorganic bases. For example, salts formed from addition of inorganic bases, include, but are not limited to alkali metal salts, such as: sodium, lithium, or potassium salts and salts formed from addition of organic bases, include, but are not limited to N-tetraalkylammonium salts, such as: N-tetrabutylammonium salt Similarly, when a compound of this invention contains a basic moiety at R₃, R⁴, R₅, R₆, R₇, R₈, R₉, or R₁₀, salts may be formed from addition of organic and inorganic acids. For example salts can be formed from the addition of acids, including but not limited to, acetic, propionic, lactic, citric, tartaric, succinic, fumaric, maleic, malonic, mandelic, malic, phthalic, hydrochloric, hydrobromic, phosphoric, nitric, sulfuric, methanesulfonic, napthalenesulfonic, benzenesulfonic, toluenesulfonic, camphorsulfonic, and similarly known acceptable acids.

In one embodiment, the present invention provides a method of inhibiting the Hepatitis C RNA-dependent RNA polymerase NS5B. The method comprises contacting a cell with an amount of a compound effective to decrease or prevent NS5B function. The cell may be a mammalian cell and more specifically a human cell. The cell may also be a bacterial cell such as for example E coli. The cell may include but is not limited to, a neuronal cell, an endothelial cell, a glial cell, a microglial cell, a smooth muscle cell, a somatic cell, a bone marrow cell, a liver cell, an intestinal cell, a germ cell, a myocyte, a mononuclear phagocyte, an endothelial cell, a tumor cell, a lymphocyte cell, a mesangial cell, a retinal epithelial cell, a retinal vascular cell, a ganglion cell or a stem cell. The cell may be a normal cell, an activated cell, a neoplastic cell, a diseased cell, or an infected cell.

In another embodiment, the present invention provides a method for the treatment or prevention of Hepatitis C infection in a mammal. The present invention accordingly provides to a mammal, a pharmaceutical composition that comprises a compound of this invention in combination or association with a pharmaceutically acceptable carrier. The compound of this invention may be administered alone or in combination with other therapeutically effective compounds or therapies for the treatment or prevention of Hepatitis C viral infection in a mammal.

The compounds are preferably provided orally or subcutaneously. The compounds may be provided by intralesional, intraperitoneal, intramuscular or intravenous injection; infusion; liposome-mediated delivery, topical, nasal, anal, vaginal, sublingual, uretheral, transdermal, intrathecal, ocular or otic delivery. In order to obtain consistency in providing the compound of this invention it is preferred that a compound of the invention is in the form of a unit dose. Suitable unit dose forms include tablets, capsules and powders in sachets or vials. Such unit dose forms may contain from 0.1 to 100 mg of a compound of the invention and preferably from 2 to 50 mg. Still further preferred unit dosage forms may contain 5 to 25 mg of a compound of the present invention. The compounds of the present invention may be administered orally at a dose range of about 0.01 to 100 mg/kg or preferably at a dose range of 0.1 to 10 mg/kg. Such compounds may be administered from 1 to 6 times a day, more usually from 1 to 4 times a day. The effective amount will be known to one of skill in the art; it will also be dependent upon the form of the compound. One of skill in the art may routinely perform empirical activity tests to determine the bioactivity of the compound in bioassays and thus determine what dosage to administer. The dosages will depend on absorption, distribution, metabolism, and excretion rates of the components of the pharmaceutical composition as well as other factors known to one of skill in the art. It is to be noted that dosage values of the pharmaceutical composition will also vary with the severity of the condition to be alleviated. It is to be further understood that for any particular subject, specific dosage regimens and schedules should be adjusted over time according to the individual's need and the professional judgment of the person administering or supervising the administration of the pharmaceutical compositions.

The compounds of the invention may be formulated with conventional excipients, such as a filler, a disintegrating agent, a binder, a lubricant, a flavoring agent, a color additive, or a carrier. The carrier may be for example a diluent, an aerosol, a topical carrier, an aqueous solution, a nonaqueous solution or a solid carrier. The carrier may be a polymer or a toothpaste. A carrier in this invention encompasses any of the standard pharmaceutically accepted carriers, such as phosphate buffered saline solution, acetate buffered saline solution, water, emulsions such as an oil/water emulsion or a triglyceride emulsion, various types of wetting agents, tablets, coated tablets and capsules.

When provided orally or topically, such compounds may be provided to a subject by delivery in different carriers. Typically, such carriers contain excipients such as starch, milk, sugar, certain types of clay, gelatin, stearic acid, talc, vegetable fats or oils, gums, or glycols. The specific carrier is selected based upon the desired method of delivery, for example, phosphate buffered saline (PBS) may be used for intravenous or systemic delivery and vegetable fats, creams, salves, ointments or gels may be used for topical delivery.

The pharmaceutical compound of the present invention may be delivered together with suitable diluents, preservatives, solubilizers, emulsifiers, adjuvants and/or carriers useful in treatment or prevention of Hepatitis C viral infection. Such compositions are liquids or lyophilized or otherwise dried formulations and include diluents of various buffer content (for example, Tris-HCl, acetate, phosphate), pH and ionic strength, additives such as albumins or gelatin to prevent absorption to surfaces, detergents (for example, TWEEN 20, TWEEN 80, PLURONIC F68, bile acid salts), solubilizing agents (for example, glycerol, polyethylene glycerol), anti-oxidants (for example ascorbic acid, sodium metabisulfate), preservatives (for example, thimerosal, benzyl alcohol, parabens), bulking substances or tonicity modifiers (for example, lactose, mannitol), covalent attachment of polymers such as polyethylene glycol, complexation with metal ions, or incorporation of the compound into or onto particulate preparations of hydrogels or liposomes, micro-emulsions, micelles, unilamellar or multilamellar vesicles, erythrocyte ghosts, or spheroplasts. Such compositions may influence the physical state, solubility, stability, rate of in vivo release, and rate of in vivo clearance of the compound or composition. The choice of compositions depends on the physical and chemical properties of the compound capable of treating or preventing a Hepatitis C viral infection.

The pharmaceutical composition of the present invention may be delivered locally via a capsule that allows a sustained release of the compound over a period of time. Controlled or sustained release compositions include formulation in lipophilic depots (for example, fatty acids, waxes, oils).

The present invention further provides controlled-release therapeutic dosage forms for the pharmaceutical composition in which the composition is incorporated into a delivery system. The dosage form controls release of the pharmaceutical composition in such a manner that an effective concentration of the composition in the blood is maintained over an extended period of time, but also the release of the composition is such that the concentration in the blood remains relatively constant over the extended period of time to improve therapeutic results and/or minimize side effects. Additionally, a controlled release system affects minimal peak to trough fluctuations in blood plasma levels of the pharmaceutical composition.

The present invention further provides a compound of the invention for use as an active therapeutic substance for preventing Hepatitis C infection. Compounds of formula (I) are of particular use for the treatment of infection with Hepatitis C virus.

The present invention further provides a method of treating Hepatitis C infection in humans, which comprises administering to the infected individual an effective amount of a compound or of a pharmaceutical composition of the invention.

The present invention also provides a pharmaceutical composition comprising a compound of a formula: 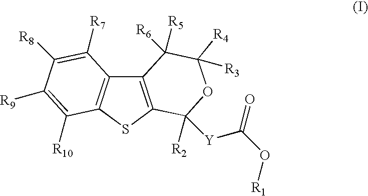
wherein:
R₁ is H, a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, an alkynyl of 2 to 7 carbon atoms, or an arylalkyl or an alkylaryl of 7 to 12 carbon atoms;
R₂ is H, a straight chain alkyl of 1 to 12 carbon atoms, a branched alkyl of 3 to 12 carbon atoms, a cycloalkyl of 3 to 12 carbon atoms, an alkenyl of 2 to 7 carbon atoms, an alkynyl of 2 to 7 carbon atoms, an alkoxyalkyl or alkoxycarbonyl of 2 to 12 carbon atoms, an arylalkyl or alkylaryl of 7 to 12 carbon atoms, a cyanoalkyl of 1 to 8 carbon atoms, an alkylthioalkyl of 2 to 16 carbon atoms, a cycloalkyl-alkyl of 4 to 24 carbon atoms, a substituted or unsubstituted aryl, or a heteroaryl;
R₃-R₆ are independently H, a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, a substituted or unsubstituted aryl, furanylmethyl, arylalkyl or alkylaryl of 7 to 12 carbon atoms, alkynyl of 2 to 7 carbon atoms, or R₅ and R₆ together with the ring carbon atom to which they are attached form a carbonyl group;
R₇-R₁₀ are independently H, a straight chain alkyl of 1 to 8 carbon atoms, a branched alkyl of 3 to 12 carbons atoms, a cycloalkyl of 3 to 12 carbon atoms, an alkenyl of 2 to 7 carbon atoms, a substituted or unsubstituted aryl, a substituted or unsubstituted heteroaryl, furanylmethyl, arylalkyl or alkylaryl of 7 to 12 carbon atoms, alkynyl of 2 to 7 carbon atoms, phenylalkynyl, alkoxy of 1 to 8 carbon atoms, arylalkoxy of 7 to 12 carbon atoms, alkylthio of 1 to 8 carbon atoms, trifluoromethoxy, trifluoroethoxy, trifluoromethylthio, trifluoroethylthio, acyl of 1 to 7 carbon atoms, COOH, COO-alkyl, CONR₁₁R₁₂, F, Cl, Br, I, CN, CF₃, NO₂, alkylsulfinyl of 1 to 8 carbon atoms, alkylsulfonyl of 1 to 6 carbon atoms, pyrrolidinyl, or thiazolidinyl;
R₁₁-R₁₂ are independently H, straight chain alkyl of 1 to 8 carbon atoms, branched alkyl of 3 to 12 carbon atoms, cycloalkyl of 3 to 12 carbon atoms, a substituted or unsubstituted aryl or heteroaryl;
Y is (CH₂)ₙ wherein n is an integer from 0 to 3, aryl or heteroaryl, cycloalkyl or heterocycloalkyl, or R₂ and Y together with the ring carbon atom to which they are attached may additionally form a spirocyclic cycloalkyl or spirocyclic heterocycloalkyl ring of 3 to 8 carbon atoms; and a pharmaceutically acceptable carrier.

In a further embodiment, this invention also provides a pharmaceutical composition comprising a compound, or a pharmaceutically acceptable salt thereof, of the formula: 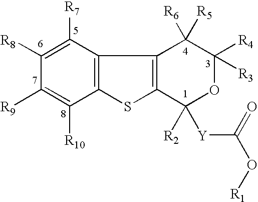
wherein:
R₁ is H; R₂ is H, a straight chain alkyl of 1 to 4 carbon atoms, a branched alkyl of 3 carbons, aryl or an ethoxyoxoethyl; R₃-R₆ are H; R₇-R₁₀ are independently H, CN, F, Cl, Br, or methyl; Y is (CH₂)ₙ wherein n is 0-3, aryl or heteroaryl, cycloalkyl or heterocycloalkyl, or R₂ and Y together with the ring carbon atom to which they are attached may additionally form a spirocyclic cycloalkyl or spirocyclic heterocycloalkyl ring of 3 to 8 carbon atoms; and a pharmaceutically acceptable carrier. In a more specific embodiment the composition contains a compound wherein R₂ is H, methyl ethyl, n-propyl, isopropyl, n-butyl, —CH₂CO₂Et or phenyl; R₇ is H, Cl, Br or CN; R₈ is H or F; R₉ is H; R₁₀ is H, Cl or methyl; Y is (CH₂)ₙ, phenyl or cyclopropyl, wherein n is an integer from 1 to 3, or Y together with R₂ forms a spirocyclic cyclohexyl; and pharmaceutically acceptable carrier.

In a preferred embodiment of the pharmaceutical composition of this invention, the compound may be selected from the group consisting of:
(1-methyl-3,4-dihydro-1H-[1]benzothieno[2,3-c]pyran-1-yl)acetic acid
3-(3,4-dihydro-1-methyl-1H-[1]benzothieno[2,3-c]pyran-1-yl)propanoic acid
4-(3,4-dihydro-1-methyl-1H-[1]benzothieno[2,3-c]pyran-1-yl)butanoic acid
(5-cyano-8-methyl-1-propyl-3,4-dihydro-1H-[1]benzothieno[2,3-c]pyran-1-yl)acetic acid; [(1R)-5-cyano-8-methyl-1-propyl-3,4-dihydro-1H-[1]benzothieno[2,3-c]pyran-1-yl]acetic acid;
[(1S)-5-cyano-8-methyl-1-propyl-3,4-dihydro-1H-[1]benzothieno[2,3-c]pyran-1-yl]acetic acid;
(5-cyano-6-fluoro-8-methyl-1-propyl-3,4-dihydro-1H-[1]benzothieno[2,3-c]pyran-1-yl)acetic acid;
[(1R)-5-cyano-6-fluoro-8-methyl-1-propyl-3,4-dihydro-1H-[1]benzothieno[2,3-c]pyran-1-yl]acetic acid;
[(1S)-5-cyano-6-fluoro-8-methyl-1-propyl-3,4-dihydro-1H-[1]benzothieno[2,3-c]pyran-1-yl]acetic acid;
(5-bromo-8-methyl-1-propyl-3,4-dihydro-1H-[1]benzothieno[2,3-c]pyran-1-yl)acetic acid;
(5,8-dichloro-1-propyl-3,4-dihydro-1H-[1]benzothieno[2,3-c]pyran-1-yl)acetic acid;
(1-butyl-5,8-dichloro-3,4-dihydro-1H-[1]benzothieno[2,3-c]pyran-1-yl)acetic acid,
(5,8-dichloro-1-ethyl-3,4-dihydro-1H-[1]benzothieno[2,3-c]pyran-1-yl)acetic acid;
(6-fluoro-8-methyl-1-propyl-3,4-dihydro-1H-[1]benzothieno[2,3-c]pyran-1-yl)acetic acid;
(1-ethyl-3,4-dihydro-1H-[1]benzothieno[2,3-c]pyran-1-yl)acetic acid;
(1-propyl-3,4-dihydro-1H-[1]benzothieno[2,3-c]pyran-1-yl)acetic acid;
(1-butyl-3,4-dihydro-1H-[1]benzothieno[2,3-c]pyran-1-yl)acetic acid;
(1-phenyl-3,4-dihydro-1H-[1]benzothieno[2,3-c]pyran-1-yl)acetic acid;
(1-isopropyl-3,4-dihydro-1H-[1]benzothieno[2,3-c]pyran-1-yl)acetic acid;
[1-(2-ethoxy-2-oxoethyl)-3,4-dihydro-1H-[1]benzothieno[2,3-c]pyran-1-yl]acetic acid;
2-(3,4-dihydro-1H-[1]benzothieno[2,3-c]pyran-1-yl)benzoic acid;
[1-(3,4-dihydro-1H-[1]benzothieno[2,3-c]pyran-1-yl)spirocyclo cyclohexyl]-acetic acid;
2-(3,4-dihydro-1H-[1]benzothieno[2,3-c]pyran-1-yl)cyclopropanecarboxylic acid, and
(5,8-dichloro-1-methyl-3,4-dihydro-1H-[1]benzothieno[2,3-c]pyran-1-yl)acetic acid.

This invention provides a compound of a formula: 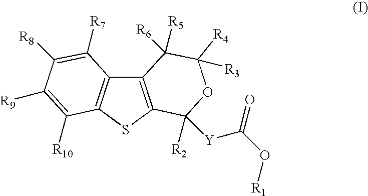
wherein:
R₁ is H, a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, an alkynyl of 2 to 7 carbon atoms, or an arylalkyl or an alkylaryl of 7 to 12 carbon atoms;
R₂ is H, a straight chain alkyl of 1 to 12 carbon atoms, a branched alkyl of 3 to 12 carbon atoms, a cycloalkyl of 3 to 12 carbon atoms, an alkenyl of 2 to 7 carbon atoms, an alkynyl of 2 to 7 carbon atoms, an alkoxyalkyl or alkoxycarbonyl of 2 to 12 carbon atoms, an arylalkyl or alkylaryl of 7 to 12 carbon atoms, a cyanoalkyl of 1 to 8 carbon atoms, an alkylthioalkyl of 2 to 16 carbon atoms, a cycloalkyl-alkyl of 4 to 24 carbon atoms, a substituted or unsubstituted aryl, or a heteroaryl;
R₃-R₆ are independently H, a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, a substituted or unsubstituted aryl, furanylmethyl, arylalkyl or alkylaryl of 7 to 12 carbon atoms, alkynyl of 2 to 7 carbon atoms, or R₅ and R₆ together with the ring carbon atom to which they are attached form a carbonyl group;
R₇-R₁₀ are independently H, a straight chain alkyl of 1 to 8 carbon atoms, a branched alkyl of 3 to 12 carbons atoms, a cycloalkyl of 3 to 12 carbon atoms, an alkenyl of 2 to 7 carbon atoms, a substituted or unsubstituted aryl, a substituted or unsubstituted heteroaryl, furanylmethyl, arylalkyl or alkylaryl of 7 to 12 carbon atoms, alkynyl of 2 to 7 carbon atoms, phenylalkynyl, alkoxy of 1 to 8 carbon atoms, arylalkoxy of 7 to 12 carbon atoms, alkylthio of 1 to 8 carbon atoms, trifluoromethoxy, trifluoroethoxy, trifluoromethylthio, trifluoroethylthio, acyl of 1 to 7 carbon atoms, COOH, COO-alkyl, CONR₁₁R₁₂, F, Cl, Br, I, CN, CF₃, NO₂, alkylsulfinyl of 1 to 8 carbon atoms, alkylsulfonyl of 1 to 6 carbon atoms, pyrrolidinyl, or thiazolidinyl;
R₁₁-R₁₂ are independently H, straight chain alkyl of 1 to 8 carbon atoms, branched alkyl of 3 to 12 carbon atoms, cycloalkyl of 3 to 12 carbon atoms, a substituted or unsubstituted aryl or heteroaryl; and
Y is (CH₂)ₙ wherein n is an integer from 1 to 3, aryl or heteroaryl, cycloalkyl or heterocycloalkyl, or R₂ and Y together with the ring carbon atom to which they are attached may additionally form a spirocyclic cycloalkyl or spirocyclic heterocycloalkyl ring of 3 to 8 carbon atoms; or a pharmaceutically acceptable salt thereof.

In an embodiment of the compound of the present invention, R₁ is H; R₂ is H, a straight chain alkyl of 1 to 4 carbon atoms, a branched alkyl of 3 carbons, aryl, or an ethoxyoxoethyl; R₃-R₆ are H; R₇-R₁₀ are independently H, CN, F, Cl, Br, or methyl; Y is (CH₂)ₙ wherein n is an integer from 1 to 3, aryl or heteroaryl, cycloalkyl or heterocycloalkyl, or R₂ and Y together with the ring carbon atom to which they are attached may additionally form a spirocyclic cycloalkyl or spirocyclic heterocycloalkyl ring of 3 to 8 carbon atoms. In a more specific embodiment R₂ is H, methyl, ethyl, n-propyl, isopropyl, n-butyl, —CH₂CO₂Et or phenyl; R₇ is H, Cl, Br or CN; R₈ is H or F; R₉ is H; R₁₀ is H, Cl or methyl; Y is (CH₂)ₙ, phenyl or cyclopropyl, wherein n is an integer from 1 to 3, or Y together with R₂ forms a spirocyclic cyclohexyl ring, or a pharmaceutically acceptable salt thereof.

In further embodiments of the compound of the present invention, the compound may be selected from any of the compounds described supra.

This invention further provides a method of treating or preventing a Hepatitis C viral infection in a mammal comprising providing the mammal with a therapeutically effective amount of a compound, wherein the compound is selected from the group of compounds described supra.

This invention also provides a method of inhibiting replication of a Hepatitis C virus comprising contacting the Hepatitis C virus with a compound, wherein the compound is selected from the group of compounds described supra.

This invention also provides a pharmaceutical composition comprising a compound, or a pharmaceutically acceptable salt thereof, of the formula: 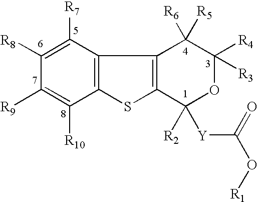
wherein:
R₁ is H; R₂ is methyl; R₃-R₆ are H; R₇-R₁₀ are independently H or Cl; Y is (CH₂)ₙ wherein n is an integer from 0 to 3; and a pharmaceutically acceptable carrier.

In a preferred embodiment of the above pharmaceutical composition of this invention, the compound may be selected from the group consisting of:
(5,8-dichloro-1-methyl-3,4-dihydro-1H-[1]benzothieno[2,3-c]pyran-1-yl)acetic acid;
(1-methyl-3,4-dihydro-1H-[1]benzothieno[2,3-c]pyran-1-yl)acetic acid;
3-(3,4-dihydro-1-methyl-1H-[1]benzothieno[2,3-c]pyran-1-yl)propanoic acid; and
4-(3,4-dihydro-1-methyl-1H-[1]benzothieno[2,3-c]pyran-1-yl)butanoic acid.

This invention also provides in another embodiment, a compound of the formula: 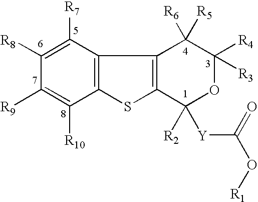
wherein:
R₁ is H; R₂ is methyl; R₃-R₆ are H; R₇-R₁₀ are independently H or Cl; and Y is (CH₂)ₙ wherein n is an integer from 0 to 3; or a pharmaceutically acceptable salt thereof.

In further embodiments of the above invention the compound may be selected from the group consisting of:
(5,8-dichloro-1-methyl-3,4-dihydro-1H-[1]benzothieno[2,3-c]pyran-1-yl)acetic acid;
(1-methyl-3,4-dihydro-1H-[1]benzothieno[2,3-c]pyran-1-yl)acetic acid;
3-(3,4-dihydro-1-methyl-1H-[1]benzothieno[2,3-c]pyran-1-yl)propanoic acid; and
4-(3,4-dihydro-1-methyl-1H-[1]benzothieno[2,3-c]pyran-1-yl)butanoic acid.

The invention further provides a method of treating or preventing a Hepatitis C viral infection in a mammal comprising providing the mammal with a therapeutically effective amount of the above disclosed compound, or a pharmaceutically acceptable salt thereof, or a pharmaceutical composition including such compound, or a pharmaceutically acceptable salt thereof. In a further embodiment of the invention the compound is selected from the group of compounds consisting of:
(5,8-dichloro-1-methyl-3,4-dihydro-1H-[1]benzothieno[2,3-c]pyran-1-yl)acetic acid;
(1-methyl-3,4-dihydro-1H-[1]benzothieno[2,3-c]pyran-1-yl)acetic acid;
3-(3,4-dihydro-1-methyl-1H-[1]benzothieno[2,3-c]pyran-1-yl)propanoic acid; and
4-(3,4-dihydro-1-methyl-1H-[1]benzothieno[2,3-c]pyran-1-yl)butanoic acid.

The invention further provides a method of inhibiting replication of a Hepatitis C virus comprising contacting the Hepatitis C virus with the above-disclosed compound or a pharmaceutical composition including such compound. In a further embodiment of the invention the compound is selected from the group of compounds consisting of:
(5,8-dichloro-1-methyl-3,4-dihydro-1H-[1]benzothieno[2,3-c]pyran-1-yl)acetic acid;
(1-methyl-3,4-dihydro-1H-[1]benzothieno[2,3-c]pyran-1-yl)acetic acid;
3-(3,4-dihydro-1-methyl-1H-[1]benzothieno[2,3-c]pyran-1-yl)propanoic acid; and
4-(3,4-dihydro-1-methyl-1H-[1]benzothieno[2,3-c]pyran-1-yl)butanoic acid.

In accordance with this invention, any of the compounds described above may be of a crystalline form or a pharmaceutically acceptable salt thereof.

The following experimental details are set forth to aid in an understanding of the invention, and are not intended, and should not be construed, to limit in any way the invention set forth in the claims that follow thereafter.

### DETAILED DESCRIPTION OF THE INVENTION

The compounds of the present invention can be readily prepared according to the following reaction schemes or modification thereof. In the following reaction schemes R₁, R₂, R₃, R₄, R₅, R₆, R₇, R₈, R₉, R₁₀, R₁₁, R₁₂ and Y are selected from the groups defined above.

Preferred compounds of the present invention can be synthesized as described in the schemes below (Scheme 1-3).

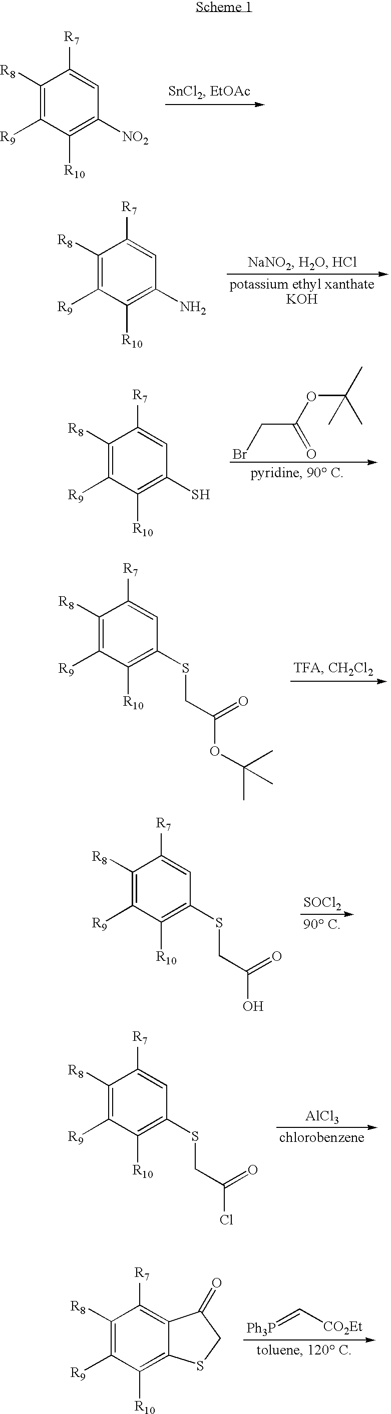

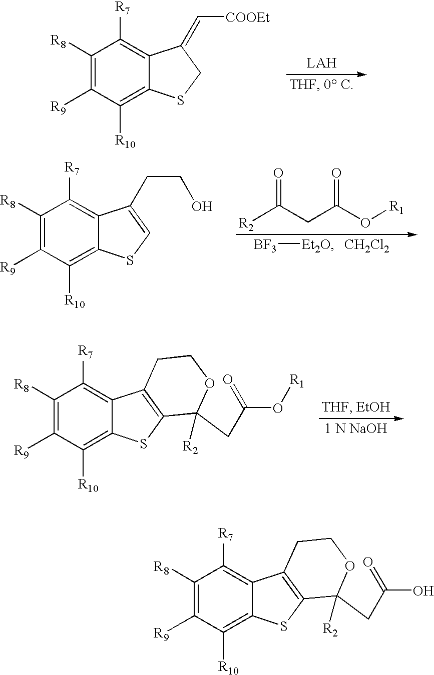

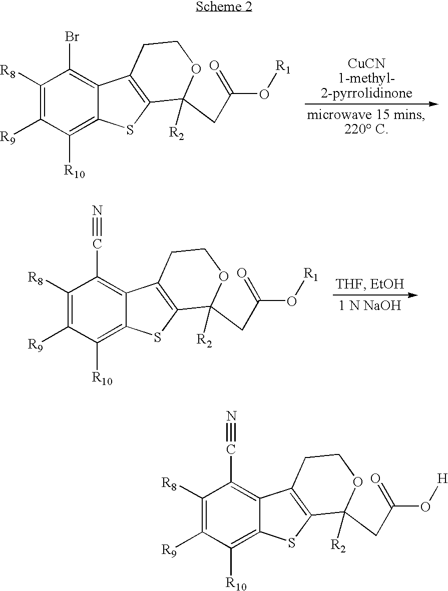

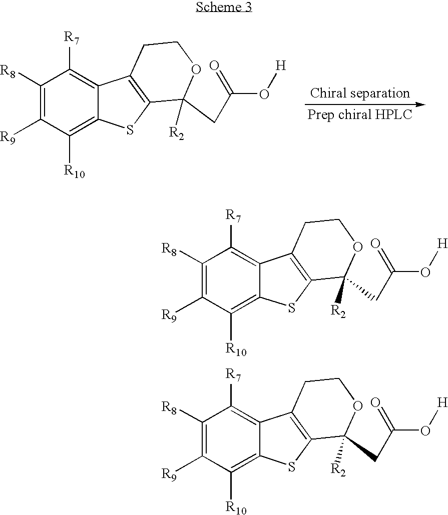

The ability of the compounds of the present invention to inhibit Hepatitis C Polymerase was established by the following experimental procedure:

NS5B from the BK strain (genotype 1b) is expressed in E. coli as a protein in which the 21 C-terminal amino acids are replaced with a short linker and a hexahistidine tag (GSHHHHHH). The purified protein is mixed with radioactive nucleotides and allowed to replicate a heteropolymeric RNA substrate, primed by an endogenous short hairpin, resulting in an approximately 760 nt product. The radioactive product is captured on a filter and quantitated after removal of the unincorporated nucleotides.

Reagents:

10 mM uridine 5′-triphosphate (UTP) (Promega # p116B)

10 mM adenine 5′-triphosphate (ATP) (Promega # p113B)

10 mM cytidine 5′-triphosphate (CTP) (Promega # p114B)

10 mM guanine 5′-triphosphate (GTP) (Promega # p115B)

Bovine Serum Albumin (BSA) 10 mg/ml NEB (100× at 10 mg/ml) #007-BSA

RNase in (Promega #N251X) 40 U/μl

A-[33P]-GTP (NEN-easytides NEG/606H 3000 Ci/mmol, 370 MBq/ml, 10 mCi/ml)

Falcon polypropylene 96 well plates (Becton Dickinson # 351190)

Millipore Multiscreen assays system-96 well-filtration plate #MADE NOB 50

Optiphase Supermix (Wallac) formulated by Fisher

Millipore Multiscreen liner for use in microbeta 1450-106 cassette [(Wallac) Perkin Elmer #1450-433]

1 M (N-[2-hydroxyethyl]piperazine-N′-[2-ethanesulfonic acid]) (HEPES), pH 7.3

Amersham Pharmacia Biotec (US16924-500 ml)

1 M MgCl₂ (SIGMA #M1028)

Dithiothreitol (DTT) (solid) (SIGMA # D9779)

RNase free water (GIBCO-BRL #10977-023)

Dimethyl sulfoxide (Aldrich #27685-5)

Basilen Blue (Sigma, B5520)

0.5M ethylenediaminetetraacetic acid (EDTA), pH 8 (GIBCO-BRL #15575-020)

Dibasic sodium phosphate (7-hydrate) (Na₂HPO₄.7H₂O; Baker#3824-07)

Phosphoric acid (Baker, #0262.02)

Further Reagent Preparation:

0.5 M Na Phosphate buffer. Per liter, weigh 134 gr Na₂HPO₄.7H₂O, add water to 900 ml. Adjust pH to 7.0 with phosphoric acid. Top off with water to 1 L.

Dilute nucleotides 1:1000 to 10 μM (GTP and CTP) or 1:100 to 100 μM (ATP and UTP) into RNase free water.

Procedure:

(1) Compounds 10 μl at 10 μg/ml in 15% Dimethylsulfoxide (DMSO)

When starting from 100 μg/ml compound stock in 1% DMSO:

Dispense 5 μl 30% DMSO per well

Dispense 5 μl compound (100 μg/ml) per well.

When starting from 50 μg/ml compound stock in 15% DMSO:

Add 10 μl compound per well.

(2) Enzyme Mix:

| | Final Conc | | | | | |
|---|---|---|---|---|---|---|
| | (in 50 μl | | Per 20 μl mix | | Per 600 | |
| Stock | assay volume) | | (1 reaction) | | reactions | |
| DEPC H₂0 | | | 17.06 | μl | 10236 | μl |
| 1 M HEPES, | 20 | mM | 0.5 | μl | 300 | μl |
| pH 7.5 | | | | | | |
| 1 M MgCl₂ | 5 | mM | 0.25 | μl | 150 | μl |
| 100 mM DTT | 1 | mM | 0.5 | μl | 300 | μl |
| 100 μM UTP | 0.5 | μM | 0.25 | μl | 150 | μl |
| 100 μM ATP | 1 | μM | 0.5 | μl | 300 | μl |
| 10 μM CTP | 0.08 | μM | 0.4 | μl | 240 | μl |
| 10 μM GTP | 0.025 | μM | 0.125 | μl | 75 | μl |
| BSA, 10 mg/ml | 0.05 | mg/ml | 0.25 | μl | 150 | μl |
| HCV RdRp | 24 | nM | 0.16 | μl | 96 | μl |
| NS5B d21BK | | | | | | |
| (500 μg/ml | | | | | | |
| or ˜7.5 μM) | | | | | | |

| | | | |
|---|---|---|---|
| Total: 20 | μl | 12 | ml |

Add 20 μl enzyme mix into each well of the assay plate. Incubate compound and enzyme at room temperature for 15 minutes.

(3) Template Mix—Prepare Ahead

Spin down a tube of RNA (5 μg/tube stored in 75% ethanol and 0.3 M sodium acetate) in a microcentrifuge for 20 minutes at 4° C. One tube is enough for 1-1.5 plates. Remove as much ethanol from the tube as possible by inverting the tube. Be gentle, pellet RNA may not adhere to the tube. Vacuum dry the RNA. Resuspend the RNA by adding 1 ml of DEPC water, close the cap of the tube tightly. To dissolve RNA, incubate RNA solution on ice for ˜60 minutes and gently vortex. Spin briefly to ensure all RNA solution is down to the bottom of the tube before opening cap. Gently transfer RNA solution into a 5 ml or larger tube. Add another 3 ml of DEPC water (total 4 ml of volume).

Add the following volumes of reagents

| | | | |
|---|---|---|---|
| | Final | Per 20 μl mix | Per 600 |
| Stock | concentration | (1 reaction) | reactions |

| | | | | | | |
|---|---|---|---|---|---|---|
| RNAse-free | | | 2.98 | μl | 1788 | μl |
| water | | | | | | |
| HEPES, 1M | 20 | mM | 0.5 | μl | 300 | μl |
| RNase | 0.4 | μ/μl | 0.5 | μl | 300 | μl |
| Inhibitor | | | | | | |
| (40 U/μl) | | | | | | |
| 33P-GTP | 0.025 | μM | 0.0125 | μl | 7.5 | μl |
| 3000 | | | | | | |
| Ci/mmol, | | | | | | |
| 10 μCi/μl | | | | | | |
| (3.3 μM) | | | | | | |
| POF RNA | 3 | nM | 16 | μl | 9600 | μl |
| template | | | | | | |

Add 20 μl template mix per reaction (i.e. 20 ng of pOF per reaction or ˜3 nM).
(4) Incubate reaction at room temperature (22-25° C.) for 2 hours.
(5) Stop reaction by adding 50 μl of 170 mM EDTA.

Final concentration of EDTA is 85 mM.

(6) Prewet filters of Millipore multiscreen assay plate by adding 200 μJ of 0.5 M sodium phosphate buffer, pH 7.0 into each well. Let stand at room temperature for 2-3 minutes.

(7) Place the multiscreen filter plate onto a Millipore Manifold and turn on vacuum to allow buffer to flow through. Turn off vacuum. Transfer 80 μl of the reaction product into each well of the filter plate. Let stand for 2-3 minutes. Turn on vacuum to filter reaction product.

(8) Turn off vacuum. Add 200 μl of 0.5 M sodium phosphate buffer, pH 7.0 into each well to wash filter. Turn on vacuum.

Repeat step (8) three more times.

(9) Remove polypropylene bottom. Spot dry filter at the bottom with paper towel. Air dry filter plate on a bench for 1 hour. Add 40 μl Super Mix scintillant. Seal top of the plate with a tape. Place plate into a Packard carrier or micro-beta carrier.

(10) Count plate using a Packard Topcount or micro-beta counter. Count (for example using Program 10) for ³³P in Top count or ³³P program in micro-beta.

Percent inhibition is calculated after background subtraction as a percent reduction of activity relative to the positive control (average value of the plate excluding the negative controls). For the primary screen hits were chosen as showing ≧75% inhibition.

See. Ferrari et al. 1999. J. Virology 73:1649-1654: “Characterization of soluble Hepatitis C virus RNA-dependent RNA polymerase expressed in E. coli and Takamizawa et all 1991” and J. Virology 65:1105-1113: “Structure and characterization of the Hepatitis C virus genome isolated from human carriers,” both references are hereby incorporated by reference in their entireties, specifically methods of testing inhibition of Hepatitis C Polymerase with test compounds.

The compounds of the present invention inhibited Hepatitis C polymerase as summarized in Table 1 A and B:

TABLE 1A

| | HCV pol | HCV pol |
|---|---|---|
| Example | BK IC₅₀ (μM) | % inh at 20 μM |
| 1 | 0.18 | |
| 2 | 17.7 | |
| 3 | 0.62 | |
| 4 | 1.9 | |
| 5 | >20 | <10 |
| 6 | 0.28 | |
| 7 | 0.32 | |
| 8 | 0.17 | |
| 9 | 2.9 | |
| 10 | 0.13 | |
| 11 | 0.016 | |
| 12 | 5.4 | |
| 13 | >20 | <10 |
| 14 | >20 | <10 |
| 15 | >20 | <10 |
| 16 | >20 | <10 |
| 17 | >20 | 27 |
| 18 | >20 | <10 |
| 19 | >20 | <10 |

The ability of the compounds of the present invention to inhibit Hepatitis C virus replicon constitutively expressed in a human liver cell line was established by the following experimental procedure:

Clone A cells (licensed from Apath, LLC) are derived from Huh-7 cells (human hepatoma cell line) and constitutively express the HCV replication proteins with concomitant amplification the HCV replicon (1b) genome. Cells are maintained and passaged in DMEM/10% FCS/1 mg/ml G418 (Geneticin from Gibco #11811-023; other media components as described below in “Elisa media”). Care is taken to maintain cell monolayers at a subeonfluent state by 1:3 or 1:4 passages every 3-4 days. The replicon is extremely sensitive to the cellular metabolism/proliferation state and replicon copy number will rapidly decline in confluent monolayers (resting cells). Under ideal conditions each cell has, on average, 1000 copies of the HCV replicon genome.

Reagents:

Elisa Media:

Dulbecco's Modified Eagle Media (DMEM) (Gibco #12430-047)

2% Fetal Calf Serum (FCS) (HyClone #SH30070.03)

1× pen-strep (Gibco #15140-122)

1× Non-essential amino acids (NEAA) (Gibco #11140-050)

no G418

Glutaraldehyde Fisher #02957-4)

TWEEN-20, 10% (Roche #1332465)

TRITON X-100 (Sigma #T-8787)

Superblock in Phosphate Buffered Saline (PBS) (Pierce #37515)

NS5a monoclonal antibody (Virostat #1873)

Goat antimouse-HRP monoclonal antibody (BioRad #172-1011)

3,3′,5,5′ tetramethylbenzidine (TMB) substrate (Sigma #T-0440)

Compound Dilution/Cell Plating:

Drug Plate Preparation (Mother Plate)

10 μl of compounds (in DMSO) are added to column 3 of the mother plate. 5 μl of DMSO are added to the remaining columns. Mother plates are set aside until ready for serial dilution to be performed.

Control Drugs

Drug and Cell Addition:

The process for each plate involves:

Prepare cell plates (daughter plates) by adding 52 μl of Elisa media to each well.

In Mother plates, serially transfer 50 μl/well from column 3 through column 12.

Transfer 8 μl from mother plate to daughter plates (all 96 wells).

Place daughter plates in incubator until cells are prepared.

Harvest Clone A cells and plate directly into daughter plates at 0.7×10⁵ cells/ml, 100 μl/well.

All plates are incubated at 37° C. in 5% CO₂ for 3 days.

Elisa Assay:

Remove media from 96-well plates (cells should be ca 80% confluent) by flicking into sink.

Add 130 μl/well 1×PBS+0.05% glutaraldehyde.

Incubate 37° C. for 1 hour.

Remove by flicking into sink.

Wash 3× with 300 μl/well PBS, shaking 5 minutes each wash. Remove by flicking into sink

Add 130 μl/well PBS+0.05% TWEEN-20+0.1% TRITON X-100.

Incubate 37° C. for 10 minutes.

Remove by flicking into sink.

Add 300 μl/well Superblock in PBS.

Incubate 37° C. for 1 hour.

Remove by flicking into sink.

Wash 3× with 300 μl/well PBS, shaking 5 minutes each wash. Remove by flicking into sink.

During last wash, make a 1:100 dilution of NS5a Monoclonal-antibody (Mab) in Superblock+0.02% TWEEN-20.

After last wash, add 50 μl/well diluted Mab.

Incubate 37° C. for 1 hour.

Remove by flicking into sink

Wash 3× with 300 μl/well PBS+0.02% TWEEN-20, shaking 5 minutes each wash.

Remove by flicking into sink.

During last wash, make a 1:500 dilution of goat antimouse-HRP Mab in Superblock+0.02% TWEEN-20.

After last wash, add 50 μl/well diluted Mab.

Incubate 37° C. for 1 hour.

Remove by flicking into sink.

Wash 5× with 300 μl/well PBS+0.02% TWEEN-20, shaking 5 minutes each wash. Remove by flicking into sink.

Wash 3× with 300 μl/well PBS, shaking 5 minutes each wash. Remove by flicking into sink.

After last wash, add 130 μl/well room temperature TMB substrate.

Incubate until blue color develops.

Add 130 μl/well 1N HCl to stop reaction (color turns from blue to yellow).

Read plates with optical density (O.D.) 450 filter.

ANALYSIS OF RESULTS: IC₅₀ (μM); IC₅₀ (μg/ml); % Inhibition

REFERENCE COMPOUNDS: Interferon-a₂; 4-30 U/ml IC50

The following non-limiting specific examples are included to illustrate the synthetic procedures used for preparing compounds of the formula (I). In these examples, all chemicals and intermediates are either commercially available or can be prepared by standard procedures found in the literature or are known to those skilled in the art of organic synthesis.

### EXAMPLE 1

### 5-Bromo-8-methyl-1-propyl-3,4-dihydro-1H-benzothieno[2,3-c]pyran-1-yl]acetic acid

### 5-Bromo-2-methylaniline

4-bromo-2-nitrotoluene (25.0 g, 0.1157 mol) in EtOAc (250 mL) was cooled to 0° C. Tin (II) chloride dihydrate (87.76 g, 0.4623 mol) was then added portionwise over 10 min while stirring. The reaction mixture was allowed to come to room temperature and then refluxed at 80° C. for 4 h. The mixture was cooled to 0° C. and neutralized with 5 N NaOH while stirring. EtOAc layer was filtered and remaining portions were extracted with EtOAc. The organic solution was washed with brine, dried with Na₂SO₄, and concentrated. The residue was purified by flash chromatography (silica, 10% EtOAC in hexanes) to give 17.367 g (80.7%) of the aniline. ¹H NMR (CDCl₃): 300 MHz δ 6.88 (m, 1H), 6.81 (m, 2H), 3.63 (bs, 2H), 2.09 (s, 3H).

### 5Bromo-2-methylbenzenethiol

A solution of sodium nitrite (6.42 g, 0.0930 mol) in water (18 mL) was added over 30 min to an ice cooled solution of 5-bromo-2-methylaniline (17.302 g, 0.0930 mol) in 35%. HC (14.2 mL, 0.1860 mol) containing ice, while potassium ethyl xanthate was prepared by rapid stirring of a mixture of KOH (6.26 g, 0.1116 mol), ethanol (13.0 mL), water (23.3 mL), and carbon disulfide (12.75 g, 0.1674 mol) for 2.5 h. The prepared potassium ethyl xanthate was slowly added to the solution of 5-bromo-2-methylbenzenediazonium salt at 0° C. After the addition, the mixture was heated at 50-55° C. for 30 min, cooled, and then extracted with ether. The organic phase was dried with Na₂SO₄ and concentrated. The residue was refluxed with KOH (27.9 g, 0.4976 mol) in ethanol for 10 h, neutralized with 10% HCl, and extracted with ether. The organic phase was dried with Na₂SO₄, concentrated, and the residue was purified by flash chromatography (silica, 100% hexanes) to give 13.75 g (72.7%) of the 5-bromo-2-methylbenzenethiol.

### (5Bromo-2-methyl-phenylsulfanyl)-acetic acid tert-butyl ester

Tert-butylbromoacetate (7.73 mL, 0.0533 mol) was added dropwise to a stirred solution of 5-bromo-2-methylbenzenethiol (10.629 g, 0.0533 mol) in pyridine (30 mL) at room temperature and allowed to stir for 15 min and then refluxed at 90° C. for 2 h. Excess pyridine was removed and the mixture was extracted with EtoAc, washed with H₂O, and 10% HCl. The organic phase was dried with Na₂SO₄, concentrated, and the residue was purified by flash chromatography (silica, 100% hexanes) to give 9.919 g (59.8%) of the (5-bromo-2-methyl-phenylsulfanyl)acetic acid tert-butyl ester. ¹H NMR (CDCl₃): 300 MHz δ 7.42 (s, 1H), 7.28 (m, 1H), 7.03 (d, J=7.89 Hz, 1H), 0.57 (s, 2H), 2.38 (s, 3H), 1.44 (s, 9H).

### (5Bromo-2-methyl-phenylsulfanyl)-acetic acid

To a solution of (5-Bromo-2-methyl-phenylsulfanyl)-acetic acid tert-butyl ester (9.919 g, 0.0313 mol) in dichloromethane (50 mL) was added trifluoroacetic acid (24.0 mL, 0.3127 mol) and the mixture stirred at room temperature for 4 h. After removal of the solvent, the residue was extracted with EtOAC and washed with H₂O. ¹H NMR (CDCl₃): 300 MHz δ 7.45 (s, 1H), 7.28 (m, 1H), 7.07 (d, J=8.13 Hz, 1H), 5.91 (bs, 1H), 3.68 (s, 2H), 2.35 (s, 3H).

### (5Bromo-2-methyl-phenylsulfanyl)-acetyl chloride

To a stirred solution of (5-Bromo-2-methyl-phenylsulfanyl)-acetic acid in 1,2-dichloroethane (25 mL) was added SOCl₂ (6.0 mL) and a catalytic amount of DMF (5-10 drops). After stirring for 4 h at 50° C., solvent was removed and residue was washed with 1,2-dichloroethane and again dried to give (5-Bromo-2-methyl-phenylsulfanyl)-acetyl chloride.

### 4-Bromo-7-methyl-benzo[b]thiophen-3-one

Aluminum chloride (7.95 g, 0.0596 mol) was added portionwise to a stirred solution of (5-bromo-2-methyl-phenylsulfanyl)-acetyl chloride in 1,2-dichloroethane (25 mL) at 0° C. After stirring for 2 h at 0° C. and 12 h at room temperature, the reaction mixture was quenched with ice. The mixture was extracted with dichloromethane, washed with H₂O and brine. The organic phase was dried with Na₂SO₄, concentrated, and the residue was purified by flash chromatography (silica, 5-10% EtOAc in hexanes) to give 2.41 g (52.0%) of the 4-bromo-7-methyl-benzo[b]thiophen-3-one. ¹H NMR (CDCl₃): 300 MHz δ 7.33 (m, 1H), 6.89 (m, 1H), 3.84 (s, 2H), 2.30 (s, 3H).

### (4-Bromo-7-trimethyl-benzo[b]thiophen-3-ylidene)-acetic acid ethyl ester

To a stirred solution of 4-bromo-7-methyl-benzo[b]thiophen-3-one (3.52 g, 0.01448 mol) in toluene (25 mL) was added (carbethoxymethylene)-triphenylphosphorane (6.06 g, 0.0174 mol). After refluxing at 120° C. for 5 d, the mixture was extracted with EtOAc, washed with H₂O and brine. The organic phase was dried with Na₂SO₄, concentrated, and the residue was purified by flash chromatography (silica, 5-10% EtOAc in hexanes) to give 1.939 g (42.7%) 4-Bromo-7-methyl-benzo[b]thiophen-3-yl)-acetic acid ethyl ester. ¹H NMR (CDCl₃): 300 MHz δ 7.46 (d, J=7.71 Hz, 1H), 7.33 (s, 1H), 6.99 (d, J=7.62 Hz, 1H), 4.20 (m, 4H), 2.51 (s, 3H), 1.27 (t, J=6.18 Hz, 3H).

### 2-(4-Bromo-7-methyl-benzo[b]thiophen-3-yl)-ethanol

To an ice-cooled solution of (4-Bromo-7-methyl-benzo[b]thiophen-3-yl)-acetic acid ethyl ester (0.783 g, 2.5 mmol) in THF (10 mL) was added lithium aluminum hydride (0.0949 g, 2.5 mmol). Reaction was monitored carefully by TLC. Upon completion of the reaction began the stepwise addition of H₂O (1.0 mL), NaOH (1.0 mL), H₂O (3.0 mL), and Na₂SO₄ (12.5 g) added after stirring 10 min. Mixture was stirred for 30 min, solids were filtered and filtrate was concentrated, extracted with EtOAc and washed with H₂O. The organic phase was dried with Na₂SO₄, concentrated, and the residue was purified by flash chromatography (silica, 15% EtOAc in hexanes) to give 0.489 g (72.1%) of the 2-(4-Bromo-7-methyl-benzo[b]thiophen-3-yl)ethanol. ¹H NMR (CDCl₃): 300 MHz δ 7.49 (d, J=7.68 Hz, 1H), 7.29 (s, 1H), 6.99 (d, J=7.71 Hz, 1H), 3.98 (t, J=6.48 Hz, 2H), 3.48 (t, J=6.48 Hz, 2H), 2.50 (s, 3H), 1.50 (bs, 1H).

### 5Bromo-8-methyl-1-propyl-3,4-dihydro-1H-benzothieno[2,3-c]pyran-1-yl]acetic acid ethyl ester

To a stirring solution of 2-(4-Bromo-7-methyl-benzo[b]thiophen-3-yl)-ethanol (0.649 g, 2.389 mmol) in dichloromethane (10 mL) was added ethyl butyrlacetate (1.146 mL, 7.166 mmol) and borontrifluoride diethyl etherate (0.908 mL, 7.166 mmol). After stirring 12 h, solvent was removed and the residue was purified by flash chromatography (silica, 10% EtOAc in hexanes) to give 0.790 g (80%) of the 5-Bromo-8-methyl-1-propyl-3,4-dihydro-1H-benzothieno[2,3-c]pyran-1-yl]acetic acid ethyl ester. ¹H NMR (CDCl₃): 300 MHz δ 7.43 (d, J=7.74 Hz, 1H), 6.94 (d, J=7.53 Hz, 1H), 4.09 (m, 4H), 3.35 (m, 2H), 2.99 (d, J=13.74 Hz, 1H), 2.83 (d, J=13.77 Hz, 1H), 2.46 (s, 3H), 2.14 (m, 1H), 1.88 (m, 1H), 1.44 (m, 1H), 1.22 (t, J=7.14 Hz, 3H), 1.96 (m, 1H), 0.88 (t, J=7.29 Hz, 3H).

### 5-Bromo-8-methyl-1-propyl-3,4-dihydro-1H-benzothieno[2,3-c]pyran-1-yl]acetic acid

To a solution of 5-Bromo-8-methyl-1-propyl-3,4-dihydro-1H-benzothieno[2,3-c]pyran-1-yl]acetic acid ethyl ester (0.343 g, 0.9603 mmol) in THF (5 mL) was added ethanol (2.0 mL) and 1 N NaOH (4.80 mL, 4.801 mmol). The reaction mixture was stirred at 50° C. for 4 h after which it was neutralized with 1 N HCl and extracted with EtOAc. The organic phase was dried with Na₂SO₄ and concentrated.

### EXAMPLE 2

### 5-Cyano-8-methyl-1-propyl-3,4-dihydro-1H-benzothieno[2,3-c]pyran-1-yl]acetic acid

### 5-Cyano-8-methyl-1-propyl-3,4-dihydro-1H-benzothieno[2,3-c]pyran-1-yl acetic acid ethyl ester

5-Bromo-8-methyl-1-propyl-3,4-dihydro-1H-benzothieno[2,3-c]pyran-1-yl]acetic acid ethyl ester (0.395 g, 0.9603 mmol) from example 1 and copper cyanide (0.215 g, 2.401 mmol) in 1-methyl-2-pyrrolidinone (3 mL) was combined in a microwave reaction vessel. The vessel was then heated in a microwave at 220° C. for 15 min. The reaction mixture was then diluted with water and extracted with EtOAc. The organic phase was dried with Na₂SO₄ and concentrated.

### 5-Cyano-8-methyl-1-propyl-3,4-dihydro-1H-benzothieno[2,3-c]pyran-1-yl]acetic acid

To a solution of 5-cyano-8-methyl-1-propyl-3,4-dihydro-1H-benzothieno[2,3-c]pyran-1-yl]acetic acid ethyl ester (0.343 g, 0.9603 mmol) in THF (5 mL) was added ethanol (2.0 mL) and 1 N NaOH (4.80 mL, 4.801 mmol). The reaction mixture was stirred at 50° C. for 4 h after which it was neutralized with 1 N HCl and extracted with EtOAc. The organic phase was dried with Na₂SO₄ and concentrated. ¹H NMR (CDCl₃): 300 MHz δ 7.65 (d, J=7.56 Hz, 1H), 7.21 (d, J=7.59 Hz, 1H), 4.19 (m, 2H) 3.31 (m, 2H), 3.0189 (d, J=14.91 Hz, 1H), 2.96 (d, J=14.91 Hz, 1H), 2.60 (s, 3H), 2.10 (m, 1H), 1.94 (m, 1H), 1.50 (m, 1H), 1.19 (m, 1H), 0.91 (t, J=7.29 Hz, 3H).

### EXAMPLE 3 AND EXAMPLE 4

### [(R)-5-Cyano-8-methyl-1-propyl-3,4-dihydro-1H-benzothieno[2,3-c]pyran-1-yl]acetic acid

### [(S)-5-Cyano-8-methyl-1-propyl-3,4-dihydro-1H-benzothieno[2,3-c]pyran-1-yl]acetic acid

Preparative HPLC using CHIRALPACK-AD (250×20 mm) and 5% isopropyl alcohol in heptane containing 0.1% TEA, gives the (R) and (S) enantiomers of 5-cyano-8-methyl-1-propyl-3,4-dihydro-1H-benzothieno[2,3-c]pyran-1-yl]acetic acid as white solids. The R and S enantiomers are dissolved separately in suitable solvent and injected onto a HP 1100 with spiderlink CHIRALPACK-AD (250×4.6 mm) HPLC column. The R and S enantiomers are eluted in isopropyl alcohol/heptane solvent mixture containing 0.1% TFA (10:90) at a flow rate of 1.0 mL/minute, DAD 215 nm; giving the (R enantiomer) with a different retention time measured in (minutes) from the (S enantiomer).

Example 5-8 were synthesized following the above described procedure for example 1 using the intermediate 2-(4,7-dichlorobenzo[b]thiophen-3-yl)-ethanol and reacting with β-ketoesters like ethylbutyryl acetate, methylacetoacetate, ethylpropionyl acetate and ethypentanoyl acetate. The resulting ester was hydrolyzed using 1N (aq) NaOH in THF/MeOH.

Example 9 was synthesized following the above described procedure for example 1 using the intermediate 2-(5-fluoro,7-methylbenzo[b]thiophen-3-yl)-ethanol and reacting with ethylbutyryl acetate. The resulting ester was hydrolyzed using 1N (aq) NaOH in THF/MeOH.

### EXAMPLE 10

### 5-Cyano-6-Fluoro-8-methyl-1-propyl-3,4-dihydro-1H-benzothieno[2,3-c]pyran-1-yl]acetic acid

It was synthesized following the above described procedure for example 1 using the intermediate 2-(4-bromo,5-fluoro,7-methylbenzo[b]thiophen-3-yl)-ethanol and reacting with ethylbutyryl acetate. The bromo ester was converted to cyano ester by following the procedure mentioned in example 2. The resulting ester was hydrolyzed using 1N (aq) NaOH in THF/MeOH.

### EXAMPLE 11 AND EXAMPLE 12

### [(R)-5-Cyano-7-fluoro-8-methyl-1-propyl-3,4-dihydro-1H-benzothieno[2,3-c]pyran-1-yl]acetic acid

### [(S)-5-Cyano-7-fluoro-8-methyl-1-propyl-3,4-dihydro-1H-benzothieno[2,3-c]pyran-1-yl]acetic acid

Preparative HPLC using CHIRALPACK-AD (250×20 mm) and 5% isopropyl alcohol in heptane containing 0.1% TFA, gives the (R) and (S) enantiomers of 5-cyano-7-fluoro-8-methyl-1-propyl-3,4-dihydro-1H-benzothieno[2,3-c]pyran-1-yl]acetic acid as white solids. The R and S enantiomers are dissolved separately in suitable solvent and injected onto a HP 1100 with spiderlink CHIRALPACK-AD (250×4.6 mm) HPLC column. The R and S enantiomers are eluted in isopropyl alcohol/heptane solvent mixture containing 0.1% TFA (10:90) at a flow rate of 1.0 mL/minute, DAD 215 nm; giving the (R enantiomer) with a different retention time measured in (minutes) from the (S enantiomer).

### EXAMPLE 13

### 1-Methyl-3,4-dihydro-1H-benzothieno[2,3-c]pyran-1-yl]acetic acid Benzo[b]thiophen-3-yl-acetic acid methyl ester

To a stirring solution of thianaphthene-3-acetic acid (2.0 g, 10.4 mmol) in methanol (20 ml) was added 10-15 drops concentrated H₂SO₄. The mixture was refluxed at 90° C. overnight Remove excess solvent and extract residue with ethyl acetate and wash with water. The organic solution was dried with Na₂SO₄, and concentrated. The product was carried without further purification.

### 2-Benzo[b]thiophen-3-yl-ethanol

Benzo[b]thiophen-3-yl-acetic acid methyl ester (2.14 g, 10.4 mmol) in THF (15 ml) was cooled to 0° C. LAH (0.417 g, 12.48 mmol) was added portionwise while stirring. Reaction mixture was allowed to come to room temperature overnight. Upon completion of the reaction began the stepwise addition of H₂O (1.0 mL), 1 N NaOH (1.0 mL), H₂O (3.0 mL), and Na₂SO₄ (12.5 g) added after stirring 10 min. Mixture was stirred for 30 min, solids were filtered and filtrate was concentrated, extracted with EtOAc and washed with H₂O. The organic solution was dried with Na₂SO₄ and concentrated. The residue was purified by flash chromatography (silica, 15% EtOAC in hexanes) to give 0.254 g of the tryptophol. ¹H NMR (CDCl₃): 300 MHz δ 7.86 (d, J=6.99 Hz, 1H), 7.76 (d, J=8.58 Hz, 1H), 7.36 (m, 2H), 7.26 (s, 1H), 7.21 (s, 1H), 3.97 (m, 2H), 3.13 (t, J=6.36 Hz, 2H), 2.26 (bs, 1H).

### 1-Methyl-3,4-dihydro-1H-benzothieno[2,3-c]pyran-1-yl]acetic acid

The compound was synthesized following the above described procedure for example 1 using the intermediate 2-(benzo[b]thiophen-3-yl)-ethanol and reacting with methylacetoacetate The resulting ester was hydrolyzed using 1N (aq) NaOH in THF/MeOH. ¹H NMR (CDCl₃): 300 MHz δ 7.61 (d, J=7.68 Hz, 1H), 7.42 (d, J=7.53 Hz, 1H), 7.20 (m, 2H), 4.07 (m, 1H), 3.97 (m, 1H), 3.68 (bs, 4H), 2.72 (m, 4H).

Example 14-19 were synthesized following the above described procedure for example 1 using the intermediate 2-(benzo[b]thiophen-3-yl)-ethanol and reacting with β-ketoesters like, ethylpropionyl acetate, ethylbutyryl acetate, ethypentanoyl acetate, ethyl isobutyryl acetate, ethylbenzoyl acetate and dimethyl-1,3-acetonedicarboxylate. The resulting ester was hydrolyzed using 1N (aq) NaOH in THF/MeOH.

TABLE 2
Pyranobenzothiophene derivatives

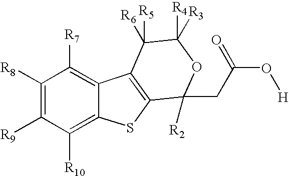

| | | | | | LC@254 | MS |
|---|---|---|---|---|---|---|
| Example | R2 | R7 | R8 | R10 | minutes | (M-H) |
| 1 | n-Pr | Br | H | CH3 | 3.35 | 382 |
| 2 | n-Pr | CN | H | CH3 | 2.91 | 328 |
| 3 | n-Pr (R) | CN | H | CH3 | 15.13 | 328 |
| 4 | n-Pr (S) | CN | H | CH3 | 17.96 | 328 |
| 5 | n-Pr | Cl | H | Cl | 3.44 | 358 |
| 6 | Me | Cl | H | Cl | 3.05 | 330 |
| 7 | n-Bu | Cl | H | Cl | 3.60 | 372 |
| 8 | Et | Cl | H | Cl | 3.21 | 344 |
| 9 | n-Pr | H | F | CH3 | 3.11 | 321 |
| 10 | n-Pr | CN | F | CH3 | 3.00 | 346 |
| 11 | n-Pr (R) | CN | F | CH3 | 14.41 | 346 |
| 12 | n-Pr (S) | CN | F | CH3 | 20.36 | 346 |
| 13 | Me | H | H | H | 2.33 | 261 |
| 14 | Et | H | H | H | 2.55 | 275 |
| 15 | n-Pr | H | H | H | 2.5 | 289 |
| 16 | n-Bu | H | H | H | 2.94 | 303 |
| 17 | Ph | H | H | H | 2.54 | 323 |
| 18 | i-Pr | H | H | H | 2.61 | 289 |
| 19 | —CH₂CO₂Et | H | H | H | 2.50 | 333 |

### EXAMPLE 1

(5-bromo-8-methyl-1-propyl-3,4-dihydro-1H-[1]benzothieno[2,3-c]pyran-1-yl)acetic acid

### EXAMPLE 2

(5-cyano-8-methyl-1-propyl-3,4-dihydro-1H-[1]benzothieno[2,3-c]pyran-1-yl)acetic acid

### EXAMPLE 3

[(1R)-5-cyano-8-methyl-1-propyl-3,4-dihydro-1H-[1]benzothieno[2,3-c]pyran-1-yl]acetic acid

### EXAMPLE 4

[(1S)-5-cyano-8-methyl-1-propyl-3,4-dihydro-1H-[1]benzothieno[2,3-c]pyran-1-yl]acetic acid

### EXAMPLE 5

(5,8-dichloro-1-propyl-3,4-dihydro-1H-[1]benzothieno[2,3-c]pyran-1-yl)acetic acid

### EXAMPLE 6

(5,8-chloro-1-methyl-3,4-dihydro-1H-[1]benzothieno[2,3-c]pyran-1-yl)acetic acid

### EXAMPLE 7

(1-butyl-5,8-dichloro-3,4-dihydro-1H-[1]benzothieno[2,3-c]pyran-1-yl)acetic acid

### EXAMPLE 8

(5,8-dichloro-1-ethyl-3,4-dihydro-1H-[1]benzothieno[2,3-c]pyran-1-yl)acetic acid

### EXAMPLE 9

(6-fluoro-8-methyl-1-propyl-3,4-dihydro-1H-[1]benzothieno[2,3-c]pyran-1-yl)acetic acid

### EXAMPLE 10

(5-cyano-6-fluoro-8-methyl-1-propyl-3,4-dihydro-1H-[1]benzothieno[2,3-c]pyran-1-yl)acetic acid

### EXAMPLE 11

[(1R)-5-cyano-6-fluoro-8-methyl-1-propyl-3,4-dihydro-1H-[1]benzothieno[2,3-c]pyran-1-yl]acetic acid

### EXAMPLE 12

[(1S)-5-cyano-6-fluoro-8-methyl-1-propyl-3,4-dihydro-1H-[1]benzothieno[2,3-c]pyran-1-yl]acetic acid

### EXAMPLE 13

(1-methyl-3,4-dihydro-1H-[1]benzothieno[2,3-c]pyran-1-yl)acetic acid

### EXAMPLE 14

(1-ethyl-3,4-dihydro-1H-[1]benzothieno[2,3-c]pyran-1-yl)acetic acid

### EXAMPLE 15

(1-propyl-3,4-dihydro-1H-[1]benzothieno[2,3-c]pyran-1-yl)acetic acid

### EXAMPLE 16

(1-butyl-3,4-dihydro-1H-[1]benzothieno[2,3-c]pyran-1-yl)acetic acid

### EXAMPLE 17

(1-phenyl-3,4-dihydro-1H-[1]benzothieno[2,3-c]pyran-1-yl)acetic acid

### EXAMPLE 18

(1-isopropyl-3,4-dihydro-1H-[1]benzothieno[2,3-c]pyran-1-yl)acetic acid

### EXAMPLE 19

[1-(2-ethoxy-2-oxoethyl)-3,4-dihydro-1H-[1]benzothieno[2,3-c]pyran-1-yl]acetic acid

What is claimed is:

1. A pharmaceutical composition comprising a compound, or a pharmaceutically acceptable salt thereof of a formula: 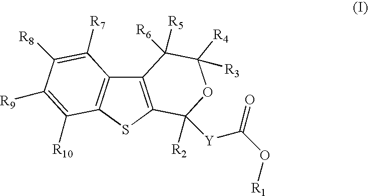
wherein:
R₁ is H, a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, an alkynyl of 2 to 7 carbon atoms, or an arylalkyl or an alkylaryl of 7 to 12 carbon atoms;
R₂ is H, a straight chain alkyl of 1 to 12 carbon atoms, a branched alkyl of 3 to 12 carbon atoms, a cycloalkyl of 3 to 12 carbon atoms, an alkenyl of 2 to 7 carbon atoms, an alkynyl of 2 to 7 carbon atoms, an alkoxyalkyl or alkoxycarbonyl of 2 to 12 carbon atoms, an arylalkyl or alkylaryl of 7 to 12 carbon atoms, a cyanoalkyl of 1 to 8 carbon atoms, an alkylthioalkyl of 2 to 16 carbon atoms, a cycloalkyl-alkyl of 4 to 24 carbon atoms, a substituted, or unsubstituted aryl, or a heteroaryl;
R₃-R₆ are independently H, a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, a substituted or unsubstituted aryl, furanylmethyl, arylalkyl or alkylaryl of 7 to 12 carbon atoms, alkynyl of 2 to 7 carbon atoms, or R₅ and R₆ together with the ring carbon atom to which they are attached form a carbonyl group;
R₇-R₁₀ are independently H, a straight chain alkyl of 1 to 8 carbon atoms, a branched alkyl of 3 to 12 carbons atoms, a cycloalkyl of 3 to 12 carbon atoms, an alkenyl of 2 to 7 carbon atoms, a substituted or unsubstituted aryl, a substituted or unsubstituted heteroaryl, furanylmethyl, arylalkyl or alkylaryl of 7 to 12 carbon atoms, alkynyl of 2 to 7 carbon atoms, phenylalkynyl, alkoxy of 1 to 8 carbon atoms, arylalkoxy of 7 to 12 carbon atoms, alkylthio of 1 to 8 carbon atoms, trifluoromethoxy, trifluoroethoxy, trifluoromethylthio, trifluoroethylthio, acyl of 1 to 7 carbon atoms, COOH, COO-alkyl, CONR₁₁R₁₂, F, Cl, Br, I, CN, CF₃, NO₂, alkylsulfinyl of 1 to 8 carbon atoms, alkylsulfonyl of 1 to 6 carbon atoms, pyrrolidinyl, or thiazolidinyl;
R₁₁-R₁₂ are independently H, straight chain alkyl of 1 to 8 carbon atoms, branched alkyl of 3 to 12 carbon atoms, cycloalkyl of 3 to 12 carbon atoms, a substituted or unsubstituted aryl or heteroaryl;
Y is (CH₂)ₙ wherein n is an integer from 0 to 3, aryl or heteroaryl, cycloalkyl or heterocycloalkyl, or R₂ and Y together with the ring carbon atom to which they are attached may additionally form a spirocyclic cycloalkyl or spirocyclic heterocycloalkyl ring of 3 to 8 carbon atoms;
and a pharmaceutically acceptable carrier.

2. The pharmaceutical composition of claim 1 wherein the compound is a crystalline form.

3. The pharmaceutical composition of claim 1 wherein the compound, or the pharmaceutically acceptable salt thereof, is the R stereoisomer, the S stereoisomer, racemic mixtures thereof, or scalemic mixtures thereof.

4. The pharmaceutical composition of claim 3 wherein the compound is a crystalline form.

5. The pharmaceutical composition of claim 1, wherein the compound, or the pharmaceutically acceptable salt thereof, has a ratio of Isomer A to Isomer B of greater than 1:1, wherein Isomer A and Isomer B have the respective formulas:

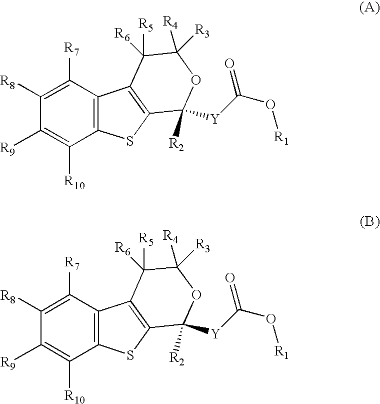

6. The pharmaceutical composition of claim 5 wherein the compound, or the pharmaceutically acceptable salt thereof, is 100% Isomer A.

7. The pharmaceutical composition of claim 5 wherein the compound, or the pharmaceutically acceptable salt thereof, has a ratio of Isomer A to Isomer B of at least about 9:1.

8. The pharmaceutical composition of claim 5 wherein the compound, or the pharmaceutically acceptable salt thereof, has a ratio of Isomer A to Isomer B of at least about 8:1.

9. The pharmaceutical composition of claim 5 wherein the compound, or the pharmaceutically acceptable salt thereof, has a ratio of Isomer A to Isomer B of at least about 7:1.

10. The pharmaceutical composition of claim 1 comprising a compound, or the pharmaceutically acceptable salt thereof, of the formula: 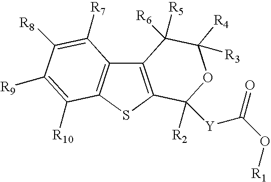
wherein:
R₁ is H;
R₂ is H, a straight chain alkyl of 2 to 4 carbon atoms, a branched alkyl of 3 carbons, aryl, or an ethoxyoxoethyl;
R₃-R₆ are H;
R₇-R₁₀ are independently H, CN, F, Cl, Br, or methyl;
Y is (CH₂)ₙ wherein n is an integer from 0 to 3, aryl or heteroaryl, cycloalkyl or heterocycloalkyl, or R₂ and Y together with the ring carbon atom to which they are attached may additionally form a spirocyclic cycloalkyl or spirocyclic heterocycloalkyl ring of 3 to 8 carbon atoms;
and a pharmaceutically acceptable carrier.

11. The pharmaceutical composition of claim 10, wherein the compound is a crystalline form.

12. The pharmaceutical composition of claim 10 comprising the compound, or the pharmaceutically acceptable salt thereof, wherein
R₂ is hydrogen, methyl, ethyl, n-propyl, isopropyl, n-butyl, —CH₂CO₂Et or phenyl;
R₉ is H;
R₇ is H, Cl, Br or CN;
R₈ is H or F;
R₁₀ is H, Cl, or CH₃; and
Y is (CH₂)ₙ, phenyl or cyclopropyl, wherein n is an integer from 1 to 3, or Y together with R₂ forms a spirocyclic cyclohexyl.

13. The pharmaceutical composition of claim 10, wherein the compound is
(5-cyano-8-methyl-1-propyl-3,4-dihydro-1H-[1]benzothieno[2,3-c]pyran-1-acetic acid, or the pharmaceutically acceptable salt thereof.

14. The pharmaceutical composition of claim 10, wherein the compound is
[(1R)-5-cyano-8-methyl-1-propyl-3,4-dihydro-1H-[1]benzothieno[2,3-c]pyran-1-yl]acetic acid, or the pharmaceutically acceptable salt thereof.

15. The pharmaceutical composition of claim 10, wherein the compound is
[(1S)-5-cyano-8-methyl-1-propyl-3,4-dihydro-1H-[1]benzothieno[2,3-c]pyran-1-yl]acetic acid, or the pharmaceutically acceptable salt thereof.

16. The pharmaceutical composition of claim 10, wherein the compound is
(5-cyano-6-fluoro-8-methyl-1-propyl-3,4-dihydro-1H-[1]benzothieno[2,3-c]pyran-1-yl)acetic acid, or the pharmaceutically acceptable salt thereof.

17. The pharmaceutical composition of claim 10, wherein the compound is
[(1R)-5-cyano-6-fluoro-8-methyl-1-propyl-3,4-dihydro-1H-[1]benzothieno[2,3-c]pyran-1-yl]acetic acid, or the pharmaceutically acceptable salt thereof.

18. The pharmaceutical composition of claim 10, wherein the compound is
[(1S)-5-cyano-6-fluoro-8-methyl-1-propyl-3,4-dihydro-1H-[1]benzothieno[2,3-c]pyran-1-yl]acetic acid, or the pharmaceutically acceptable salt thereof.

19. The pharmaceutical composition of claim 10, wherein the compound is
(5-bromo-8-methyl-1-propyl-3,4-dihydro-1H-[1]benzothieno[2,3-c]pyran-1-yl)acetic acid, or the pharmaceutically acceptable salt thereof.

20. The pharmaceutical composition of claim 10, wherein the compound is
(5,8-dichloro-1-propyl-3,4-dihydro-1H-[1]benzothieno[2,3-c]pyran-1-yl)acetic acid, or the pharmaceutically acceptable salt thereof.

21. The pharmaceutical composition of claim 10, wherein the compound is
(1-butyl-5,8-dichloro-3,4-dihydro-1H-[1]benzothieno[2,3-c]pyran-1-yl)acetic acid, or the pharmaceutically acceptable salt thereof.

22. The pharmaceutical composition of claim 10, wherein the compound is
(5,8-dichloro-1-ethyl-3,4-dihydro-1H-[1]benzothieno[2,3-c]pyran-1-yl)acetic acid, or the pharmaceutically acceptable salt thereof.

23. The pharmaceutical composition of claim 10, wherein the compound is
(6-fluoro-8-methyl-1-propyl-3,4-dihydro-1H-[1]benzothieno[2,3-c]pyran-1-yl)acetic acid, or the pharmaceutically acceptable salt thereof.

24. The pharmaceutical composition of claim 10 wherein the compound is
(5,8-dichloro-1-methyl-3,4-dihydro-1H-[1]benzothieno[2,3-c]pyran-1-yl)acetic acid, or the pharmaceutically acceptable salt thereof.

25. The pharmaceutical composition of claim 10 wherein the compound is
(1-butyl-5,8-dichloro-3,4-dihydro-1H-[1]benzothieno[2,3-c]pyran-1-yl)acetic acid, or the pharmaceutically acceptable salt thereof.

26. The pharmaceutical composition of claim 10 wherein the compound is
(1-methyl-3,4-dihydro-1H-[1]benzothieno[2,3-c]pyran-1-yl)acetic acid, or the pharmaceutically acceptable salt thereof.

27. The pharmaceutical composition of claim 10 wherein the compound is
(1-ethyl-3,4-dihydro-1H-[1]benzothieno[2,3-c]pyran-1-yl)acetic acid, or the pharmaceutically acceptable salt thereof.

28. The pharmaceutical composition of claim 10 wherein the compound is
(1-propyl-3,4-dihydro-1H-[1]benzothieno[2,3-c]pyran-1-yl)acetic acid, or the pharmaceutically acceptable salt thereof.

29. The pharmaceutical composition of claim 10 wherein the compound is
(1-butyl-3,4-dihydro-1H-[1]benzothieno[2,3-c]pyran-1-yl)acetic acid, or the pharmaceutically acceptable salt thereof.

30. The pharmaceutical composition of claim 10 wherein the compound is
(1-phenayl-3,4-dihydro-1H-[1]benzothieno[2,3-c]pyran-1-yl)acetic acid, or the pharmaceutically acceptable salt thereof.

31. The pharmaceutical composition of claim 10 wherein the compound is
(1-isopropyl-3,4-dihydro-1H-[1]benzothieno[2,3-c]pyran-1-yl)acetic acid, or the pharmaceutically acceptable salt thereof.

32. The pharmaceutical composition of claim 10 wherein the compound is
[1-(2-ethoxy-2-oxoethyl)-3,4-dihydro-1H-[1]benzothieno[2,3-c]pyran-1-yl]acetic acid, or the pharmaceutically acceptable salt thereof.

33. A pharmaceutical composition comprising a compound, or a pharmaceutically acceptable salt thereof, of a formula: 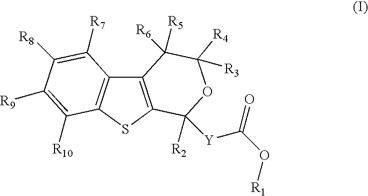
wherein:
R₁ is H;
R₂ is methyl;
R₃-R₆ are H;
R₇-R₁₀ are independently H or Cl;
Y is (CH₂)ₙ wherein n is an integer from 0 to 3;
and a pharmaceutically acceptable carrier.

34. The pharmaceutical composition of claim 33, wherein the compound is a crystalline form.

35. The pharmaceutical composition of claim 33, wherein the compound is
(5,8-dichloro-1-methyl-3,4-dihydro-1H-[1]benzothieno[2,3-c]pyran-1-yl)acetic acid, or the pharmaceutically acceptable salt thereof.

36. The pharmaceutical composition of claim 33, wherein the compound is
(1-methyl-3,4-dihydro-1H-[1]benzothieno[2,3-c]pyran-1-yl)acetic acid, or the pharmaceutically acceptable salt thereof.

37. The pharmaceutical composition of claim 33, wherein the compound is
3-(3,4-dihydro-1-methyl-1H-[1]benzothieno[2,3-c]pyran-1-yl)propanoic acid, or the pharmaceutically acceptable salt thereof.

38. The pharmaceutical composition of claim 33, wherein the compound is
4-(3,4-dihydro-1-methyl-1H-[1]benzothieno[2,3-c]pyran-1-yl)butanoic acid, or the pharmaceutically acceptable salt thereof.

39. A compound of a formula: 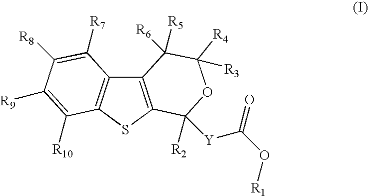
wherein:
R₁ is H, a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, an alkynyl of 2 to 7 carbon atoms, or an arylalkyl or an alkylaryl of 7 to 12 carbon atoms;
R₂ is H, a straight chain alkyl of 1 to 12 carbon atoms, a branched alkyl of 3 to 12 carbon atoms, a cycloalkyl of 3 to 12 carbon atoms, an alkenyl of 2 to 7 carbon atoms, an alkynyl of 2 to 7 carbon atoms, an alkoxyalkyl or alkoxycarbonyl of 2 to 12 carbon atoms, an arylalkyl or alkylaryl of 7 to 12 carbon atoms, a cyanoalkyl of 1 to 8 carbon atoms, an alkylthioalkyl of 2 to 16 carbon atoms, a cycloalkyl-alkyl of 4 to 24 carbon atoms, a substituted or unsubstituted aryl, or a heteroaryl;
R₃-R₆ are independently H, a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, a substituted or unsubstituted aryl, furanylmethyl, arylalkyl or alkylaryl of 7 to 12 carbon atoms, alkynyl of 2 to 7 carbon atoms, or R₅ and R₆ together with the ring carbon atom to which they are attached form a carbonyl group;
R₇-R₁₀ are independently H, a straight chain alkyl of 1 to 8 carbon atoms, a branched alkyl of 3 to 12 carbons atoms, a cycloalkyl of 3 to 12 carbon atoms, an alkenyl of 2 to 7 carbon atoms, a substituted or unsubstituted aryl, a substituted or unsubstituted heteroaryl, furanylmethyl, arylalkyl or alkylaryl of 7 to 12 carbon atoms, alkynyl of 2 to 7 carbon atoms, phenylalkynyl, alkoxy of 1 to 8 carbon atoms, alkylalkoxy of 7 to 12 carbon atoms, alkylthio of 1 to 8 carbon atoms, trifluoromethoxy, trifluoroethoxy, trifluoromethylthio, trifluoroethylthio, acyl of 1 to 7 carbon atoms, COOH, COO-alkyl, CONR₁₁R₁₂, F, Cl, Br, I, CN, CF₃, NO₂, alkylsulfinyl of 1 to 8 carbon atoms, alkylsulfonyl of 1 to 6 carbon atoms, pyrrolidinyl, or thiazolidinyl;
R₁₁-R₁₂ are independently H, straight chain alkyl of 1 to 8 carbon atoms, branched alkyl of 3 to 12 carbon atoms, cycloalkyl of 3 to 12 carbon atoms, a substituted or unsubstituted aryl or heteroaryl;
Y is (CH₂)ₙ wherein n is an integer from 0 to 3, aryl or heteroaryl, cycloalkyl or heterocycloalkyl, or R₂ and Y together with the ring carbon atom to which they are attached may additionally form a spirocyclic cycloalkyl or spirocyclic heterocycloalkyl ring of 3 to 8 carbon atoms; or
a pharmaceutically acceptable salt thereof.

40. A compound of claim 39, wherein the compound is a crystalline form.

41. The compound of claim 39 having the formula: 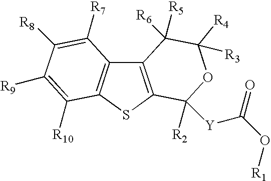
wherein:
R₁ is H;
R₂ is H, a straight chain alkyl of 1 to 4 carbon atoms, a branched alkyl of 3 carbons, aryl, or an ethoxyoxoethyl;
R₃-R₆ are H;
R₇-R₁₀ are independently H, CN, F, Cl, Br, or methyl;
Y is (CH₂)ₙ wherein n is an integer from 0 to 3, aryl or heteroaryl, cycloalkyl or heterocycloalkyl, or R₂ and Y together with the ring carbon atom to which they are attached may additionally form a spirocyclic cycloalkyl or spirocyclic heterocycloalkyl ring of 3 to 8 carbon atoms; or the pharmaceutically acceptable salt.

42. The Compound of claim 41, wherein:
R₂ is H, methyl, ethyl, n-propyl isopropyl, n-butyl, —CH₂CO₂Et or phenyl;
R₇ is H, Cl, Br or CN;
R₈ is H, Cl or methyl;
R₉ is H;
R₁₀ is H, Cl or methyl; and
Y is (CH₂)ₙ, phenyl or cyclopropyl wherein n is an integer from 1 to 3, or Y together with R₂ forms a spirocyclic cyclohexyl;
or the pharmaceutically acceptable salt thereof.

43. The compound of claim 41, wherein the compound is a crystalline form.

44. The compound of claim 41, wherein the compound is
(5-cyano-8-methyl-1-propyl-3,4-dihydro-1H-[1]benzothieno[2,3-c]pyran-1-acetic acid, or the pharmaceutically acceptable salt thereof.

45. The compound of claim 41, wherein the compound is
[(1R)-5-cyano-8-methyl-1-propyl-3,4-dihydro-1H-[1]benzothieno[2,3-c]pyran-1-yl]acetic acid, or the pharmaceutically acceptable salt thereof.

46. The compound of claim 41, wherein the compound is
[(1S)-5-cyano-8-methyl-1-propyl-3,4-dihydro-1H-[1]benzothieno[2,3-c]pyran-1-yl]acetic acid, or the pharmaceutically acceptable salt thereof.

47. The compound of claim 41, wherein the compound is
(5-cyano-6-fluoro-8-methyl-1-propyl-3,4-dihydro-1H-[1]benzothieno[2,3-c]pyran-1-yl)acetic acid, or the pharmaceutically acceptable salt thereof.

48. The compound of claim 41, wherein the compound is
[(1R)-5-cyano-6-fluoro-8-methyl-1-propyl-3,4-dihydro-1H-[1]benzothieno[2,3-c]pyran-1-yl]acetic acid, or the pharmaceutically acceptable salt thereof.

49. The compound of claim 41, wherein the compound is
[(1S)-5-cyano-6-fluoro-8-methyl-1-propyl-3,4-dihydro-1H-[1]benzothieno[2,3-c]pyran-1-yl]acetic acid, or the pharmaceutically acceptable salt thereof.

50. The compound of claim 41, wherein the compound is
(5-bromo-8-methyl-1-propyl-3,4-dihydro-1H-[1]benzothieno[2,3-c]pyran-1-yl)acetic acid, or the pharmaceutically acceptable salt thereof.

51. The compound of claim 41, wherein the compound is
(5,8-dichloro-1-propyl-3,4-dihydro-1H-[1]benzothieno[2,3-c]pyran-1-yl)acetic acid, or the pharmaceutically acceptable salt thereof.

52. The compound of claim 41, wherein the compound is
(1-butyl-5,8-dichloro-3,4-dihydro-1H-[1]benzothieno[2,3-c]pyran-1-yl)acetic acid, or the pharmaceutically acceptable salt thereof.

53. The compound of claim 41, wherein the compound is
(5,8-dichloro-1-ethyl-3,4-dihydro-1H-[1]benzothieno[2,3-c]pyran-1-yl)acetic acid, or the pharmaceutically acceptable salt thereof.

54. The compound of claim 41, wherein the compound is
(6-fluoro-8-methyl-1-propyl-3,4-dihydro-1H-[1]benzothieno[2,3-c]pyran-1-yl)acetic acid, or the pharmaceutically acceptable salt thereof.

55. The compound of claim 41, wherein the compound is
(1-ethyl-3,4-dihydro-1H-[1]benzothieno[2,3-c]pyran-1-yl)acetic acid, or the pharmaceutically acceptable salt thereof.

56. The compound of claim 41, wherein the compound is
(1-propyl-3,4-dihydro-1H-[1]benzothieno[2,3-c]pyran-1-yl)acetic acid, or the pharmaceutically acceptable salt thereof.

57. The compound of claim 41, wherein the compound is
(1-butyl-3,4-dihydro-1H-[1]benzothieno[2,3-c]pyran-1-yl)acetic acid, or the pharmaceutically acceptable salt thereof.

58. The compound of claim 41, wherein the compound is
(1-phenyl-3,4-dihydro-1H-[1]benzothieno[2,3-c]pyran-1-yl)acetic acid, or the pharmaceutically acceptable salt thereof.

59. The compound of claim 41, wherein the compound is
(1-isopropyl-3,4-dihydro-1H-[1]benzothieno[2,3-c]pyran-1-yl)acetic acid, or the pharmaceutically acceptable salt thereof.

60. The compound of claim 41, wherein the compound is
[1-(2-ethoxy-2-oxoethyl)-3,4-dihydro-1H-[1]benzothieno[2,3-c]pyran-1-yl]acetic acid, or the pharmaceutically acceptable salt thereof.

61. A compound of a formula: 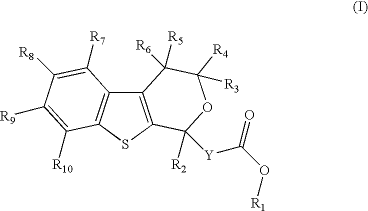
wherein:
R₁ is H;
R₂ is methyl;
R₃-R₆ are H;
R₇-R₁₀ are independently H or Cl;
Y is (CH₂)ₙ wherein n is an integer from 0 to 3;
or a pharmaceutically acceptable salt thereof.

62. The compound of claim 61, wherein the compound is a crystalline form.

63. The compound of claim 61, wherein the compound is
(5,8-dichloro-1-methyl-3,4-dihydro-1H-[1]benzothieno[2,3-c]pyran-1-yl)acetic acid, or the pharmaceutically acceptable salt thereof.

64. The compound of claim 61, wherein the compound is
(1-methyl-3,4-dihydro-1H-[1]benzothieno[2,3-c]pyran-1-yl)acetic acid, or the pharmaceutically acceptable salt thereof.

65. A method of obtaining the compounds of formulas (A) and (B): 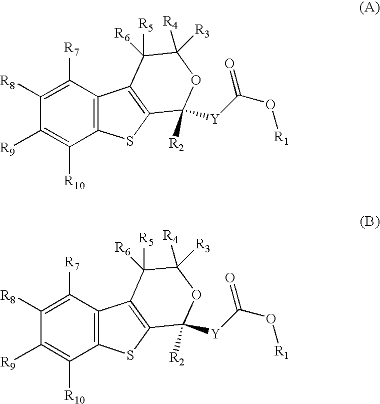
wherein:
R₁ is H, a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, an alkynyl of 2 to 7 carbon atoms, or an arylalkyl or an alkylaryl of 7 to 12 carbon atoms;
R₂ is H, a straight chain alkyl of 1 to 12 carbon atoms, a branched alkyl of 3 to 12 carbon atoms, a cycloalkyl of 3 to 12 carbon atoms, an alkenyl of 2 to 7 carbon atoms, an alkynyl of 2 to 7 carbon atoms, an alkoxyalkyl or alkoxycarbonyl of 2 to 12 carbon atoms, an arylalkyl or alkylaryl of 7 to 12 carbon atoms, a cyanoalkyl of 1 to 8 carbon atoms, an alkylthioalkyl of 2 to 16 carbon atoms, a cycloalkyl-alkyl of 4 to 24 carbon atoms, a substituted or unsubstituted aryl, or a heteroaryl;
R3-R6 are independently H, a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, a substituted or unsubstituted aryl, furanylmethyl, arylalkyl or alkylaryl of 7 to 12 carbon atoms, alkynyl of 2 to 7 carbon atoms, or R₅ and R₆ together with the ring carbon atom to which they are attached form a carbonyl group;
R₇-R₁₀ are independently H, a straight chain alkyl of 1 to 8 carbon atoms, a branched alkyl of 3 to 12 carbons atoms, a cycloalkyl of 3 to 12 carbon atoms, an alkenyl of 2 to 7 carbon atoms, a substituted or unsubstituted aryl, a substituted or unsubstituted heteroaryl, furanylmethyl, arylalkyl or alkylaryl of 7 to 12 carbon atoms, alkynyl of 2 to 7 carbon atoms, phenylalkynyl, alkoxy of 1 to 8 carbon atoms, arylalkoxy of 7 to 12 carbon atoms, alkylthio of 1 to 8 carbon atoms, trifluoromethoxy, trifluoroethoxy, trifluoromethylthio, trifluoroethylthio, acyl of 1 to 7 carbon atoms, COOH, COO-alkyl, CONR₁₁R₁₂, F, Cl, Br, I, CN, CF₃, NO₂, alkylsulfinyl of 1 to 8 carbon atoms, alkylsulfonyl of 1 to 6 carbon atoms, pyrrolidinyl, or thiazolidinyl;
R₁₁-R₁₂ are independently H, straight chain alkyl of 1 to 8 carbon atoms, branched alkyl of 3 to 12 carbon atoms, cycloalkyl of 3 to 12 carbon atoms, a substituted or unsubstituted aryl or heteroaryl;
Y is (CH₂)ₙ wherein n is an integer from 0 to 3, aryl or heteroaryl, cycloalkyl or heterocycloalkyl, or R₂ and Y together with the ring carbon atom to which they are attached may additionally form a spirocyclic cycloalkyl or spirocyclic heterocycloalkyl ring of 3 to 8 carbon atoms; and
said method comprising the steps of:
(a) adding a concentrated solution of a racemic mixture of the compound to a chiral High Performance Liquid Chromatrography (HPLC) column;
(b) eluting the (R) and (S) enantiomers from the column in step (a) with isopropyl alcohol and heptane solvent containing TFA;
(c) drying the (R) and (S) enantiomers separately,
(d) dissolving the (R) and (S) enantiomers from step (c) separately in a suitable solvent;
(e) injecting the dissolved (R) and (S) enantiomers from step (d) separately onto chiral HPLC column;
(f) eluting the respective (R) and (S) enantiomers at a rate of 1.0 ml/minute from the column in step (e) with isopropyl alcohol and heptane solvent containing TFA, wherein the (R) enantiomer has a different retention time from the (S) enantiomer and each respective enantiomer is detected by its absorption at 215 nm;
(g) combining the (R) enantiomer eluants from step (f) and drying the (R) enantiomer to obtain the (R) enantiomer compound; and
(h) combining the (S) enantiomer eluants from step (f) and drying the (S) enantiomer to obtain the (S) enantiomer compound.

66. A method of treating or preventing a Hepatitis C viral infection in a mammal comprising the steps of providing the mammal with a therapeutically effective amount of a compound of a formula: 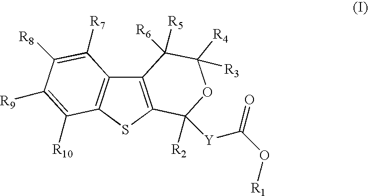
wherein:
R₁ is H, a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, an alkynyl of 2 to 7 carbon atoms, or an arylalkyl or an alkylaryl of 7 to 12 carbon atoms;
R₂ is H, a straight chain alkyl of 1 to 12 carbon atoms, a branched alkyl of 3 to 12 carbon atoms, a cycloalkyl of 3 to 12 carbon atoms, an alkenyl of 2 to 7 carbon atoms, an alkynyl of 2 to 7 carbon atoms, an alkoxyalkyl or alkoxycarbonyl of 2 to 12 carbon atoms, an arylalkyl or alkylaryl of 7 to 12 carbon atoms, a cyanoalkyl of 1 to 8 carbon atoms, an alkylthioalkyl of 2 to 16 carbon atoms, a cycloalkyl-alkyl of 4 to 24 carbon atoms, a substituted or unsubstituted aryl, or a heteroaryl;
R₃-R₆ are independently H, a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, a substituted or unsubstituted aryl, furanylmethyl, arylalkyl or alkylaryl of 7 to 12 carbon atoms, alkynyl of 2 to 7 carbon atoms, or R₅ and R₆ together with the ring carbon atom to which they are attached form a carbonyl group;
R₇-R₁₀ are independently H, a straight chain alkyl of 1 to 8 carbon atoms, a branched alkyl of 3 to 12 carbons atoms, a cycloalkyl of 3 to 12 carbon atoms, an alkenyl of 2 to 7 carbon atoms, a substituted or unsubstituted aryl, a substituted or unsubstituted heteroaryl, furanylmethyl, arylalkyl or alkylaryl of 7 to 12 carbon atoms, alkynyl of 2 to 7 carbon atoms, phenylalkynyl, alkoxy of 1 to 8 carbon atoms, arylalkoxy of 7 to 12 carbon atoms, alkylthio of 1 to 8 carbon atoms, trifluoromethoxy, trifluoroethoxy, trifluoromethylthio, trifluoroethylthio, acyl of 1 to 7 carbon atoms, COOH, COO-alkyl, CONR₁₁R₁₂, F, Cl, Br, I, CN, CF₃, NO₂, alkylsulfinyl of 1 to 8 carbon atoms, alkylsulfonyl of 1 to 6 carbon atoms, pyrrolidinyl, or thiazolidinyl;
R₁₁-R₁₂ are independently H, straight chain alkyl of 1 to 8 carbon atoms, branched alkyl of 3 to 12 carbon atoms, cycloalkyl of 3 to 12 carbon atoms, a substituted or unsubstituted aryl or heteroaryl;
Y is (CH₂)ₙ wherein n is an integer from 0 to 3, aryl or heteroaryl, cycloalkyl or heterocycloalkyl, or R₂ and Y together with the ring carbon atom to which they are attached may additionally form a spirocyclic cycloalkyl or spirocyclic heterocycloalkyl ring of 3 to 8 carbon atoms;
or a pharmaceutically acceptable salt thereof.

67. The method of claim 66, wherein the compound is a crystalline form.

68. The method of claim 66, wherein the compound, or the pharmaceutically acceptable salt thereof, has a ratio of Isomer A to Isomer B of greater than 1:1, wherein Isomer A and Isomer B have the respective formulas:

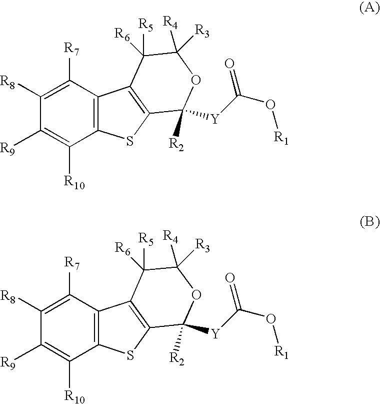

69. The method of claim 68, wherein the compound, or the pharmaceutically acceptable salt thereof, is 100% Isomer A.

70. The method of claim 68, wherein the compound, or the pharmaceutically acceptable salt thereof, has a ratio of Isomer A to Isomer B of at least about 9:1.

71. The method of claim 68, wherein the compound, or the pharmaceutically acceptable salt thereof, or the pharmaceutically acceptable salt thereof, has a ratio of Isomer A to Isomer B of at least about 8:1.

72. The method of claim 68, wherein the compound, or the pharmaceutically acceptable salt thereof or the pharmaceutically acceptable salt thereof, has a ratio of Isomer A to Isomer B of at least about 7:1.

73. The method of claim 66, wherein the compound has the formula: 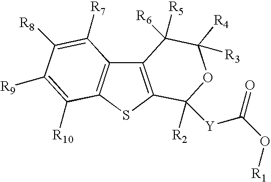
wherein:
R₁ is H;
R₂ is H, a straight chain alkyl of 1 to 4 carbon atoms, a branched alkyl of 3 carbons, aryl or an ethoxyoxoethyl;
R₃-R⁶ are H;
R₇-R₁₀ are independently H, CN, F, Cl, Br, or methyl;
Y is (CH₂)ₙ wherein n is an integer from 0 to 3, aryl or heteroaryl, cycloalkyl or heterocycloalkyl, or R₂ and Y together with the ring carbon atom to which they are attached may additionally form a spirocyclic cycloalkyl or spirocyclic heterocycloalkyl ring of 3 to 8 carbon atoms;
or the pharmaceutically acceptable salt thereof.

74. The method of claim 73 comprising the compound, or the pharmaceutically acceptable salt thereof, wherein:
R₂ is H, methyl, ethyl, n-propyl, isopropyl, n-butyl, —CH₂CO₂Et or phenyl;
R₇ is H, Cl, Br or CN;
R₈ is H, Cl or methyl;
R₉ is H;
R₁₀ is H, Cl or methyl; and
Y is (CH₂)ₙ, phenyl or cyclopropyl, wherein n is an integer from 1 to 3, or Y together with R₂ forms a spirocyclic cyclohexyl.

75. The method of claim 73, wherein the compound is a crystalline form.

76. The method of claim 73, wherein the compound is
(5-cyano-8-methyl-1-propyl-3,4-dihydro-1H-[1]benzothieno[2,3-c]pyran-1-yl)acetic acid, or the pharmaceutically acceptable salt thereof.

77. The method of claim 73, wherein the compound is
[(1R)-5-cyano-8-methyl-1-propyl-3,4-dihydro-1H-[1]benzothieno[2,3-c]pyran-1-yl]acetic acid, or the pharmaceutically acceptable salt thereof.

78. The method of claim 73, wherein the compound is
[(1S)-5-cyano-8-methyl-1-propyl-3,4-dihydro-1H-[1]benzothieno[2,3-c]pyran-1-yl]acetic acid, or the pharmaceutically acceptable salt thereof.

79. The method of claim 73, wherein the compound is
(5-cyano-6-fluoro-8-methyl-1-propyl-3,4-dihydro-1H-[1]benzothieno[2,3-c]pyran-1-yl)acetic acid, or the pharmaceutically acceptable salt thereof.

80. The method of claim 73, wherein the compound is
[(1R)-5-cyano-6-fluoro-8-methyl-1-propyl-3,4-dihydro-1H-[1]benzothieno[2,3-c]pyran-1-yl]acetic acid, or the pharmaceutically acceptable salt thereof.

81. The method of claim 73, wherein the compound is
[(1S)-5-cyano-6-fluoro-8-methyl-1-propyl-3,4-dihydro-1H-[1]benzothieno[2,3-c]pyran-1-yl]acetic acid, or the pharmaceutically acceptable salt thereof.

82. The method of claim 73, wherein the compound is
(5-bromo-8-methyl-1-propyl-3,4-dihydro-1H-[1]benzothieno[2,3-c]pyran-1-yl)acetic acid, or the pharmaceutically acceptable salt thereof.

83. The method of claim 73, wherein the compound is
(5,8-dichloro-1-propyl-3,4-dihydro-1H-[1]benzothieno[2,3-c]pyran-1-yl)acetic acid, or the pharmaceutically acceptable salt thereof.

84. The method of claim 73, wherein the compound is
(1-butyl-5,8-dichloro-3,4-dihydro-1H-[1]benzothieno[2,3-c]pyran-1-yl)acetic acid, or the pharmaceutically acceptable salt thereof.

85. The method of claim 73, wherein the compound is
(5,8-dichloro-1-ethyl-3,4-dihydro-1H-[1]benzothieno[2,3-c]pyran-1-yl)acetic acid, or the pharmaceutically acceptable salt thereof.

86. The method of claim 73, wherein the compound is
(6-fluoro-8-methyl-1-propyl-3,4-dihydro-1H-[1]benzothieno[2,3-c]pyran-1-yl)acetic acid, or the pharmaceutically acceptable salt thereof.

87. The method of claim 73, wherein the compound is
(1-ethyl-3,4-dihydro-1H-[1]benzothieno[2,3-c]pyran-1-yl)acetic acid, or the pharmaceutically acceptable salt thereof.

88. The method of claim 73, wherein the compound is
(1-propyl-3,4-dihydro-1H-[1]benzothieno[2,3-c]pyran-1-yl)acetic acid, or the pharmaceutically acceptable salt thereof.

89. The method of claim 73, wherein the compound is
(1-butyl-3,4-dihydro-1H-[1]benzothieno[2,3-c]pyran-1-yl)acetic acid, or the pharmaceutically acceptable salt thereof.

90. The method of claim 73, wherein the compound is
(1-phenyl-3,4-dihydro-1H-[1]benzothieno[2,3-c]pyran-1-yl)acetic acid, or the pharmaceutically acceptable salt thereof.

91. The method of claim 73, wherein the compound is
(1-isopropyl-3,4-dihydro-1H-[1]benzothieno[2,3-c]pyran-1-yl)acetic acid, or the pharmaceutically acceptable salt thereof.

92. The method of claim 73, wherein the compound is
[1-(2-ethoxy-2-oxoethyl)-3,4-dihydro-1H-[1]benzothieno[2,3-c]pyran-1-yl]acetic acid, or the pharmaceutically acceptable salt thereof.

93. A method of treating or preventing a Hepatitis C viral infection in a mammal comprising providing the mammal with a therapeutically effective amount of a compound of a formula: 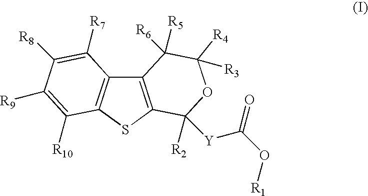
wherein:
R₁ is H;
R₂ is methyl;
R₃-R₆ are H;
R₇-R₁₀ are independently H or Cl;
Y is (CH₂)ₙ wherein n is an integer from 0 to 3;
or a pharmaceutically acceptable salt thereof.

94. The method of claim 93, wherein the compound is a crystalline form.

95. The method of claim 93, wherein the compound is
(5,8-dichloro-1-methyl-3,4-dihydro-1H-[1]benzothieno[2,3-c]pyran-1-yl)acetic acid, or the pharmaceutically acceptable salt thereof.

96. The method of claim 93, wherein the compound is
(1-methyl-3,4-dihydro-1H-[1]benzothieno[2,3-c]pyran-1-yl)acetic acid, or the pharmaceutically acceptable salt thereof.

97. A method of inhibiting replication of a Hepatitis C virus comprising contacting the Hepatitis C virus with a compound of a formula: 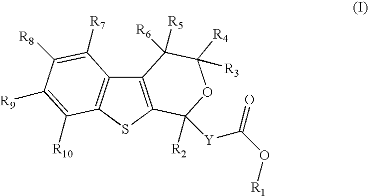
wherein:
R₁ is H, a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, an alkynyl of 2 to 7 carbon atoms, or an arylalkyl or an alkylaryl of 7 to 12 carbon atoms;
R₂ is H, a straight chain alkyl of 1 to 12 carbon atoms, a branched alkyl of 3 to 12 carbon atoms, a cycloalkyl of 3 to 12 carbon atoms, an alkenyl of 2 to 7 carbon atoms, an alkynyl of 2 to 7 carbon atoms, an alkoxyalkyl or alkoxycarbonyl of 2 to 12 carbon atoms, an arylalkyl or alkylaryl of 7 to 12 carbon atoms, a cyanoalkyl of 1 to 8 carbon atoms, an alkylthioalkyl of 2 to 16 carbon atoms, a cycloalkyl-alkyl of 4 to 24 carbon atoms, a substituted or unsubstituted aryl, or a heteroaryl;
R₃-R₆ are independently H, a straight chain alkyl of 1 to 8 carbon atoms, a branched alkyl of 3 to 12 carbon atoms, a cycloalkyl of 3 to 12 carbon atoms, an alkenyl of 2 to 7 carbon atoms, a substituted or unsubstituted aryl, furanylmethyl, arylalkyl or alkylaryl of 7 to 12 carbon atoms, alkynyl of 2 to 7 carbon atoms, or R₅ and R₆ together with the ring carbon atom to which they are attached form a carbonyl group;
R₇-R₁₀ are independently H, a straight chain alkyl of 1 to 8 carbon atoms, a branched alkyl of 3 to 12 carbons atoms, a cycloalkyl of 3 to 12 carbon atoms, an alkenyl of 2 to 7 carbon atoms, a substituted or unsubstituted aryl, a substituted or unsubstituted heteroaryl, furanylmethyl, arylalkyl or alkylaryl of 7 to 12 carbon atoms, alkynyl of 2 to 7 carbon atoms, phenylalkynyl, alkoxy of 1 to 8 carbon atoms, arylalkoxy of 7 to 12 carbon atoms, alkylthio of 1 to 8 carbon atoms, trifluoromethoxy, trifluoroethoxy, trifluoromethylthio, trifluoroethylthio, acyl of 1 to 7 carbon atoms, COOH, COO-alkyl, CONR₁₁R₁₂, F, Cl, Br, I, CN, CF₃, NO₂, alkylsulfinyl of 1 to 8 carbon atoms, alkylsulfonyl of 1 to 6 carbon atoms, pyrrolidinyl, or thiazolidinyl;
R₁₁-R₁₂ are independently H, straight chain alkyl of 1 to 8 carbon atoms, branched alkyl of 3 to 12 carbon atoms, cycloalkyl of 3 to 12 carbon atoms, a substituted or unsubstituted aryl or heteroaryl;
Y is (CH₂)ₙ wherein n is an integer from 0 to 3, aryl or heteroaryl, cycloalkyl or heterocycloalkyl, or R₂ and Y together with the ring carbon atom to which they are attached may additionally form a spirocyclic cycloalkyl or spirocyclic heterocycloalkyl ring of 3 to 8 carbon atoms;
or a pharmaceutically acceptable salt thereof.

98. The method of claim 97, wherein the compound is a crystalline form.

99. The method of claim 97, wherein the compound, or the pharmaceutically acceptable salt thereof, includes the R stereoisomer, the S stereoisomer, racemic mixtures thereof or scalemic mixtures thereof.

100. The method of claim 99, wherein the compound is a crystalline form.

101. The method of claim 97, wherein the compound, or the pharmaceutically acceptable salt thereof, has a ratio of Isomer A to Isomer B of greater than 1:1, wherein Isomer A and Isomer B have the respective formulas:

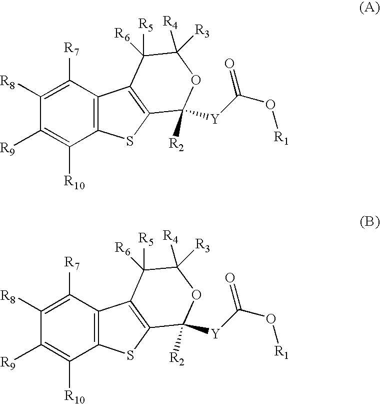

102. The method of claim 101, wherein the compound, or the pharmaceutically acceptable salt thereof, is 100% Isomer A.

103. The method of claim 101, wherein the compound, or the pharmaceutically acceptable salt thereof, has a ratio of Isomer A to Isomer B of at least about 9:1.

104. The method of claim 101, wherein the compound, or the pharmaceutically acceptable salt thereof, has a ratio of Isomer A to Isomer B of at least about 8:1.

105. The method of claim 101, wherein the compound, or the pharmaceutically acceptable salt thereof, has a ratio of Isomer A to Isomer B of at least about 7:1.

106. The method of claim 101, wherein the compound has the formula: 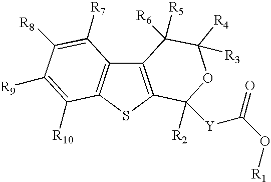
wherein:
R₁ is H;
R₂ is H, a straight chain alkyl of 1 to 4 carbon atoms, a branched alkyl of 3 carbons, aryl, or an ethoxyoxoethyl;
R₃-R₆ are H;
R₇-R₁₀ are independently H, CN, F, Cl, Br, or methyl;
Y is (CH₂)ₙ wherein n is an integer from 0 to 3, aryl or heteroaryl, cycloalkyl or heterocycloalkyl, or R₂ and Y together with the ring carbon atom to which they are attached may additionally form a spirocyclic cycloalkyl or spirocyclic heterocycloalkyl ring of 3 to 8 carbon atoms;
or the pharmaceutically acceptable salt thereof.

107. The method of claim 106, comprising the compound, or the pharmaceutically acceptable salt thereof, wherein:
R₂ is H, methyl, ethyl, n-propyl, isopropyl, n-butyl, —CH₂CO₂Et or phenyl;
R₇ is H, Cl, Br or CN;
R₈ is H, Cl or methyl;
R₉ is H;
R₁₀ is H, Cl or methyl; and
Y is (CH₂)ₙ, phenyl or cyclopropyl, wherein n is an integer from 1 to 3, or Y together with R₂ forms a spirocyclic cyclohexyl.

108. The method of claim 106, wherein the compound is a crystalline form.

109. The method of claim 106, wherein the compound is
(5-cyano-8-methyl-1-propyl-3,4-dihydro-1H-[1]benzothieno[2,3-c]pyran-1-yl)acetic acid, or the pharmaceutically acceptable salt thereof.

110. The method of claim 106, wherein the compound is
[(1R)-5-cyano-8-methyl-1-propyl-3,4-dihydro-1H-[1]benzothieno[2,3-c]pyran-1-yl]acetic acid, or the pharmaceutically acceptable salt thereof.

111. The method of claim 106, wherein the compound is
[(1S)-5-cyano-8-methyl-1-propyl-3,4-dihydro-1H-[1]benzothieno[2,3-c]pyran-1-yl]acetic acid, or the pharmaceutically acceptable salt thereof.

112. The method of claim 106, wherein the compound is
(5-cyano-6-fluoro-8-methyl-1-propyl-3,4-dihydro-1H-[1]benzothieno[2,3-c]pyran-1-yl)acetic acid, or the pharmaceutically acceptable salt thereof.

113. The method of claim 106, wherein the compound is
[(1R)-5-cyano-6-fluoro-8-methyl-1-propyl-3,4-dihydro-1H-[1]benzothieno[2,3-c]pyran-1-yl]acetic acid, or the pharmaceutically acceptable salt thereof.

114. The method of claim 106, wherein the compound is
[(1S)-5-cyano-6-fluoro-8-methyl-1-propyl-3,4-dihydro-1H-[1]benzothieno[2,3-c]pyran-1-yl]acetic acid, or the pharmaceutically acceptable salt thereof.

115. The method of claim 106, wherein the compound is
(5-bromo-8-methyl-1-propyl-3,4-dihydro-1H-[1]benzothieno[2,3-c]pyran-1-yl)acetic acid, or the pharmaceutically acceptable salt thereof.

116. The method of claim 106, wherein the compound is
(5,8-dichloro-1-propyl-3,4-dihydro-1H-[1]benzothieno[2,3-c]pyran-1-yl)acetic acid, or the pharmaceutically acceptable salt thereof.

117. The method of claim 106, wherein the compound is
(1-butyl-5,8-dichloro-3,4-dihydro-1H-[1]benzothieno[2,3-c]pyran-1-yl)acetic acid, or the pharmaceutically acceptable salt thereof.

118. The method of claim 106, wherein the compound is
(5,8-dichloro-1-ethyl-3,4-dihydro-1H-[1]benzothieno[2,3-c]pyran-1-yl)acetic acid, or the pharmaceutically acceptable salt thereof.

119. The method of claim 106, wherein the compound is
(6-fluoro-8-methyl-1-propyl-3,4-dihydro-1H-[1]benzothieno[2,3-c]pyran-1-yl)acetic acid, or the pharmaceutically acceptable salt thereof.

120. The method of claim 106, wherein the compound is
(1-ethyl-3,4-dihydro-1H-[1]benzothieno[2,3-c]pyran-1-yl)acetic acid, or the pharmaceutically acceptable salt thereof.

121. The method of claim 106, wherein the compound is
(1-propyl-3,4-dihydro-1H-[1]benzothieno[2,3-c]pyran-1-yl)acetic acid, or the pharmaceutically acceptable salt thereof.

122. The method of claim 106, wherein the compound is
(1-butyl-3,4-dihydro-1H-[1]benzothieno[2,3-c]pyran-1-yl)acetic acid, or the pharmaceutically acceptable salt thereof.

123. The method of claim 106, wherein the compound is
(1-phenyl-3,4-dihydro-1H-[1]benzothieno[2,3-c]pyran-1-yl)acetic acid, or the pharmaceutically acceptable salt thereof.

124. The method of claim 106, wherein the compound is
(1-isopropyl-3,4-dihydro-1H-[1]benzothieno[2,3-c]pyran-1-yl)acetic acid, or the pharmaceutically acceptable salt thereof.

125. The method of claim 106, wherein the compound is
[1-(2-ethoxy-2-oxoethyl)-3,4-dihydro-1H-[1]benzothieno[2,3-c]pyran-1-yl]acetic acid, or the pharmaceutically acceptable salt thereof.

126. A method of inhibiting replication of a Hepatitis C virus comprising contacting the Hepatitis C virus with a compound of a formula: 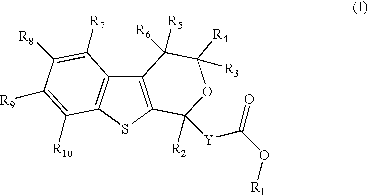
wherein:
R₁ is H;
R₂ is methyl;
R₃-R₆ are H;
R₇-R₁₀ are independently H or Cl;
Y is (CH₂)ₙ wherein n is an integer from 0 to 3;
or a pharmaceutically acceptable salt thereof.

127. The method of claim 126, wherein the compound is a crystalline form.

128. The method of claim 126, wherein the compound is
(5,8-dichloro-1-methyl-3,4-dihydro-1H-[1]benzothieno[2,3-c]pyran-1-yl)acetic acid, or the pharmaceutically acceptable salt thereof.

129. The method of claim 126, wherein the compound is
(1-methyl-3,4-dihydro-1H-[1]benzothieno[2,3-c]pyran-1-yl)acetic acid, or the pharmaceutically acceptable salt thereof.
